SEQUENCE LISTING (159 sequences)

SEQ ID NO: 1 - protein, 10 aa, Artificial Sequence
  DENV-DC1
TGQNSRLKRS

SEQ ID NO: 2 - protein, 10 aa, Artificial Sequence
  DENV-DC2
TGQMSRNKRS

SEQ ID NO: 3 - protein, 10 aa, Artificial Sequence
  DENV-DC3
TGSNYRSKRS

SEQ ID NO: 4 - protein, 10 aa, Artificial Sequence
  DENV-DC4
TGLFTRNKRS

SEQ ID NO: 5 - protein, 10 aa, Artificial Sequence
  DENV-DC5
TGRLRRAKRS

SEQ ID NO: 6 - protein, 10 aa, Artificial Sequence
  DENV-DC6
TGTPKRLKRS

SEQ ID NO: 7 - protein, 10 aa, Artificial Sequence
  DENV-DC7
TGKINRAKRS

SEQ ID NO: 8 - protein, 10 aa, Artificial Sequence
  DENV-DC8
TGSFTRSKRS

SEQ ID NO: 9 - protein, 10 aa, Artificial Sequence
  DENV-DC9
TGGSPRAKRS

SEQ ID NO: 10 - protein, 10 aa, Artificial Sequence
  DENV-DC10
TGSKLRIKRS

SEQ ID NO: 11 - protein, 10 aa, Artificial Sequence
  DENV-DC11
TGTGTRLKRS

SEQ ID NO: 12 - protein, 10 aa, Artificial Sequence
  DENV-DC12
TGHMNRLKRS

SEQ ID NO: 13 - protein, 10 aa, Artificial Sequence
  DENV-DC13
TGFSTRQKRS

SEQ ID NO: 14 - protein, 10 aa, Artificial Sequence
  DENV-DC14
TGGTTRAKRS

SEQ ID NO: 15 - protein, 10 aa, Artificial Sequence
  DENV-DC15
TGESMRSKRS

SEQ ID NO: 16 - protein, 10 aa, Artificial Sequence
  DENV-DC16
TGYRSRPKRS

SEQ ID NO: 17 - protein, 10 aa, Artificial Sequence
  DENV-DC17
TGSNSRAKRS

SEQ ID NO: 18 - protein, 10 aa, Artificial Sequence
  DENV-DC18
TGRSIRSKRS

SEQ ID NO: 19 - protein, 10 aa, Artificial Sequence
  DENV-DC19
TGHDSRHKRS

SEQ ID NO: 20 - protein, 10 aa, Artificial Sequence
  DENV-DC20
TGFVGRHKRS

SEQ ID NO: 21 - protein, 10 aa, Artificial Sequence
  DENV-DC21
TGGAHRLKRS

SEQ ID NO: 22 - protein, 10 aa, Artificial Sequence
  DENV-DC22
TGSNPRMKRS

SEQ ID NO: 23 - protein, 10 aa, Artificial Sequence
  DENV-DC23
TGSNTRIKRS

SEQ ID NO: 24 - protein, 10 aa, Artificial Sequence
  DENV-DC24
TGVTARTKRS

SEQ ID NO: 25 - protein, 10 aa, Artificial Sequence
  DENV-DC25
TGTRVRSKRS

SEQ ID NO: 26 - protein, 10 aa, Artificial Sequence
  DENV-DC26
TGHVGRDKRS

SEQ ID NO: 27 - protein, 10 aa, Artificial Sequence
  DENV-DC27
TGSIMRHKRS

SEQ ID NO: 28 - protein, 10 aa, Artificial Sequence
  DENV-DC28
TGPKSRLKRS

SEQ ID NO: 29 - protein, 10 aa, Artificial Sequence
  DENV-DC29
TGNVRRYKRS

SEQ ID NO: 30 - protein, 10 aa, Artificial Sequence
  DENV-DC30
TGYSRRDKRS

SEQ ID NO: 31 - protein, 10 aa, Artificial Sequence
  DENV-DC31
TGYGHRYKRS

SEQ ID NO: 32 - protein, 10 aa, Artificial Sequence
  DENV-DC32
TGAANRLKRS

SEQ ID NO: 33 - protein, 10 aa, Artificial Sequence
  DENV-DC33
TGVHNRNKRS

SEQ ID NO: 34 - protein, 10 aa, Artificial Sequence
  DENV-DC34
TGVTARLKRS

SEQ ID NO: 35 - protein, 10 aa, Artificial Sequence
  DENV-DC35
TGHNTRDKRS

SEQ ID NO: 36 - protein, 10 aa, Artificial Sequence
  DENV-DC36
TGASHRPKRS

SEQ ID NO: 37 - protein, 10 aa, Artificial Sequence
  DENV-DC37
TGGTRRVKRS

SEQ ID NO: 38 - protein, 10 aa, Artificial Sequence
  DENV-DC38
TGVPMRQKRS

SEQ ID NO: 39 - protein, 10 aa, Artificial Sequence
  DENV-DC39
TGSGNRAKRS

SEQ ID NO: 40 - protein, 10 aa, Artificial Sequence
  DENV-DC40
TGKIGREKRS

SEQ ID NO: 41 - protein, 10 aa, Artificial Sequence
  DENV-DC41
TGHHNRTKRS

SEQ ID NO: 42 - protein, 10 aa, Artificial Sequence
  DENV-DC42
TGWTARSKRS

SEQ ID NO: 43 - protein, 10 aa, Artificial Sequence
  DENV-DC43
TGSKIRDKRS

SEQ ID NO: 44 - protein, 10 aa, Artificial Sequence
  DENV-DC44
TGLHGRPKRS

SEQ ID NO: 45 - protein, 10 aa, Artificial Sequence
  DENV-DC45
TGTAGRNKRS

SEQ ID NO: 46 - protein, 10 aa, Artificial Sequence
  DENV-DC46
TGDQRRLKRS

SEQ ID NO: 47 - protein, 10 aa, Artificial Sequence
  DENV-DC47
TGRTHRFKRS

SEQ ID NO: 48 - protein, 10 aa, Artificial Sequence
  DENV-DC48
TGGFGRSKRS

SEQ ID NO: 49 - protein, 10 aa, Artificial Sequence
  DENV-DC49
TGHSYRPKRS

SEQ ID NO: 50 - protein, 10 aa, Artificial Sequence
  DENV-DC50
TGERWRHKRS

SEQ ID NO: 51 - protein, 10 aa, Artificial Sequence
  DENV-DV1
TGMAKRSKRS

SEQ ID NO: 52 - protein, 10 aa, Artificial Sequence
  DENV-DV2
TGTAKRSKRS

SEQ ID NO: 53 - protein, 10 aa, Artificial Sequence
  DENV-DV3
TGLSRRSKRS

SEQ ID NO: 54 - protein, 10 aa, Artificial Sequence
  DENV-DV4
TGRQARSKRS

SEQ ID NO: 55 - protein, 10 aa, Artificial Sequence
  DENV-DV5
TGKMRREKRS

SEQ ID NO: 56 - protein, 10 aa, Artificial Sequence
  DENV-DV6
TGSNKRHKRS

SEQ ID NO: 57 - protein, 10 aa, Artificial Sequence
  DENV-DV7
TGERARVKRS

SEQ ID NO: 58 - protein, 10 aa, Artificial Sequence
  DENV-DV8
TGRYKRDKRS

SEQ ID NO: 59 - protein, 10 aa, Artificial Sequence
  DENV-DV9
TGGKSRVKRS

SEQ ID NO: 60 - protein, 10 aa, Artificial Sequence
  DENV-DV10
TGRPVRSKRS

SEQ ID NO: 61 - protein, 10 aa, Artificial Sequence
  DENV-DV11
TGHSRREKRS

SEQ ID NO: 62 - protein, 10 aa, Artificial Sequence
  DENV-DV12
TGWGKRSKRS

SEQ ID NO: 63 - protein, 10 aa, Artificial Sequence
  DENV-DV13
TGTGRRMKRS

SEQ ID NO: 64 - protein, 10 aa, Artificial Sequence
  DENV-DV14
TGRSKRSKRS

SEQ ID NO: 65 - protein, 10 aa, Artificial Sequence
  DENV-DV15
TGSVRRVKRS

SEQ ID NO: 66 - protein, 10 aa, Artificial Sequence
  DENV-DV16
TGASHRSKRS

SEQ ID NO: 67 - protein, 10 aa, Artificial Sequence
  DENV-DV17
TGMSKRTKRS

SEQ ID NO: 68 - protein, 10 aa, Artificial Sequence
  DENV-DV18
TGFKHRVKRS

SEQ ID NO: 69 - protein, 10 aa, Artificial Sequence
  DENV-DV19
TGGRHRNKRS

SEQ ID NO: 70 - protein, 10 aa, Artificial Sequence
  DENV-DV20
TGATKRSKRS

SEQ ID NO: 71 - protein, 10 aa, Artificial Sequence
  DENV-DV21
TGISKRGKRS

SEQ ID NO: 72 - protein, 10 aa, Artificial Sequence
  DENV-DV22
TGNHRRNKRS

SEQ ID NO: 73 - protein, 10 aa, Artificial Sequence
  DENV-DV23
TGSLRRIKRS

SEQ ID NO: 74 - protein, 10 aa, Artificial Sequence
  DENV-DV24
TGQYKRSKRS

SEQ ID NO: 75 - protein, 10 aa, Artificial Sequence
  DENV-DV25
TGRPRRDKRS

SEQ ID NO: 76 - protein, 10 aa, Artificial Sequence
  DENV-DV26
TGYSKRPKRS

SEQ ID NO: 77 - protein, 10 aa, Artificial Sequence
  DENV-DV27
TGMAQRSKRS

SEQ ID NO: 78 - protein, 10 aa, Artificial Sequence
  DENV-DV28
TGTSRRNKRS

SEQ ID NO: 79 - protein, 10 aa, Artificial Sequence
  DENV-DV29
TGQKARSKRS

SEQ ID NO: 80 - protein, 10 aa, Artificial Sequence
  DENV-DV30
TGIAKRSKRS

SEQ ID NO: 81 - protein, 10 aa, Artificial Sequence
  DENV-DV31
TGGRTRQKRS

SEQ ID NO: 82 - protein, 10 aa, Artificial Sequence
  DENV-DV32
TGRKVRSKRS

SEQ ID NO: 83 - protein, 10 aa, Artificial Sequence
  DENV-DV33
TGSMKRSKRS

SEQ ID NO: 84 - protein, 10 aa, Artificial Sequence
  DENV-DV34
TGTAQRSKRS

SEQ ID NO: 85 - protein, 10 aa, Artificial Sequence
  DENV-DV35
TGNTHRTKRS

SEQ ID NO: 86 - protein, 10 aa, Artificial Sequence
  DENV-DV36
TGGFRRYKRS

SEQ ID NO: 87 - protein, 10 aa, Artificial Sequence
  DENV-DV37
TGNKSRNKRS

SEQ ID NO: 88 - protein, 10 aa, Artificial Sequence
  DENV-DV38
TGKTRRDKRS

SEQ ID NO: 89 - protein, 10 aa, Artificial Sequence
  DENV-DV39
TGMTRRGKRS

SEQ ID NO: 90 - protein, 10 aa, Artificial Sequence
  DENV-DV40
TGRHRRDKRS

SEQ ID NO: 91 - protein, 10 aa, Artificial Sequence
  DENV-DV41
TGTSRRHKRS

SEQ ID NO: 92 - protein, 10 aa, Artificial Sequence
  DENV-DV42
TGQNRRDKRS

SEQ ID NO: 93 - protein, 10 aa, Artificial Sequence
  DENV-DV43
TGMAKRTKRS

SEQ ID NO: 94 - protein, 10 aa, Artificial Sequence
  DENV-DV44
TGTAKRLKRS

SEQ ID NO: 95 - protein, 10 aa, Artificial Sequence
  DENV-DV45
TGSLSRHKRS

SEQ ID NO: 96 - protein, 10 aa, Artificial Sequence
  DENV-DV46
TGLSKRSKRS

SEQ ID NO: 97 - protein, 10 aa, Artificial Sequence
  DENV-DV47
TGASRRDKRS

SEQ ID NO: 98 - protein, 10 aa, Artificial Sequence
  DENV-DV48
TGSKNRAKRS

SEQ ID NO: 99 - protein, 10 aa, Artificial Sequence
  DENV-DV49
TGSRVRDKRS

SEQ ID NO: 100 - protein, 10 aa, Artificial Sequence
  DENV-DV50
TGDTRRSKRS

SEQ ID NO: 101 - protein, 10 aa, Artificial Sequence
  DV2-V1
TGAGRRSKRS

SEQ ID NO: 102 - protein, 10 aa, Artificial Sequence
  DV2-V2
TGSKMRSKRS

SEQ ID NO: 103 - protein, 10 aa, Artificial Sequence
  DV2-C1
TGRAQRYKRS

SEQ ID NO: 104 - protein, 166 aa, Dengue virus type 2
FHLTTRNGEPHMIVSRQEKGKSLLFKTEDGVNMCTLMAMDLGELCEDTITYNCPLLRQNEPEDIDCWCNSTSTWVTYGTCTTTGEHRREKRSVALVPHVGMGLETRTETWMSSEGAWKHAQRIETWILRHPGFTIMAAILAYTIGTTHFQRALIFILLTAVAPSMT

SEQ ID NO: 105 - DNA, 498 bp, Dengue virus type 2
ttccatctaaccacacgtaacggagaaccacacatgatcgtcagtagacaagagaaagggaaaagtcttctgtttaaaacagaggatggtgtgaacatgtgcaccctcatggccatggaccttggtgaattgtgtgaagacacaatcacgtacaactgtccccttctcaggcagaatgagccagaagacatagactgttggtgcaactccacgtccacatgggtaacttatgggacttgtactaccacgggagaacatagaagagaaaaaagatcagtggcactcgttccacatgtgggaatgggactggagacgcgaactgaaacatggatgtcatcagaaggggcttggaaacatgcccagagaattgaaacttggatcctgagacatccaggcttcaccataatggcagcaatcctggcatataccatagggacgacacatttccagagagccctgattttcatcttactgacagctgtcgctccttcaatgaca

SEQ ID NO: 106 - protein, 166 aa, Dengue virus type 1
FHLTTRGGEPHMIVSKQERGKSLLFKTSAGVNMCTLIAMDLGELCEDTMTYKCPRITETEPDDVDCWCNATETWVTYGTCSQTGEHRRDKRSVALAPHVGLGLETRTETWMSSEGAWKQIQKVETWALRHPGFTVIALFLAHAIGTSITQKGIIFILLMLVTPSMA

SEQ ID NO: 107 - protein, 166 aa, Dengue virus type 3
FHLTSRDGEPRMIVGKNERGKSLLFKTASGINMCTLIAMDLGEMCDDTVTYKCPHITEVEPEDIDCWCNLTSTWVTYGTCNQAGEHRRDKRSVALAPHVGMGLDTRTQTWMSAEGAWRQVEKVETWALRHPGFTILALFLAHYIGTSLTQKVVIFILLMLVTPSMT

SEQ ID NO: 108 - protein, 166 aa, Dengue virus type 4
FHLSTRDGEPLMIVAKHERGRPLLFKTTEGINKCTLIAMDLGEMCEDTVTYKCPLLVNTEPEDIDCWCNLTSTWVMYGTCTQSGERRREKRSVALTPHSGMGLETRAETWMSSEGAWKHAQRVESWILRNPGFALLAGFMAYMIGQTGIQRTVFFVLMMLVAPSYG

SEQ ID NO: 109 - protein, 166 aa, Artificial Sequence
  DV2-V2 prM
FHLTTRNGEPHMIVSRQEKGKSLLFKTEDGVNMCTLMAMDLGELCEDTITYNCPLLRQNEPEDIDCWCNSTSTWVTYGTCTTTGSKMRSKRSVALVPHVGMGLETRTETWMSSEGAWKHAQRIETWILRHPGFTIMAAILAYTIGTTHFQRALIFILLTAVAPSMT

SEQ ID NO: 110 - protein, 166 aa, Artificial Sequence
  DV2-C1 prM
FHLTTRNGEPHMIVSRQEKGKSLLFKTEDGVNMCTLMAMDLGELCEDTITYNCPLLRQNEPEDIDCWCNSTSTWVTYGTCTTTGRAQRYKRSVALVPHVGMGLETRTETWMSSEGAWKHAQRIETWILRHPGFTIMAAILAYTIGTTHFQRALIFILLTAVAPSMT

SEQ ID NO: 111 - protein, 166 aa, Artificial Sequence
  DV2-V1 prM
FHLTTRNGEPHMIVSRQEKGKSLLFKTEDGVNMCTLMAMDLGELCEDTITYNCPLLRQNEPEDIDCWCNSTSTWVTYGTCTTTGAGRRSKRSVALVPHVGMGLETRTETWMSSEGAWKHAQRIETWILRHPGFTIMAAILAYTIGTTHFQRALIFILLTAVAPSMT

SEQ ID NO: 112 - protein, 166 aa, Artificial Sequence
  DV1-prM-D89K
FHLTTRGGEPHMIVSKQERGKSLLFKTSAGVNMCTLIAMDLGELCEDTMTYKCPRITETEPDDVDCWCNATETWVTYGTCSQTGEHRRKKRSVALAPHVGLGLETRTETWMSSEGAWKQIQKVETWALRHPGFTVIALFLAHAIGTSITQKGIIFILLMLVTPSMA

SEQ ID NO: 113 - protein, 166 aa, Artificial Sequence
  DV2-prM-E89K
FHLTTRNGEPHMIVSRQEKGKSLLFKTEDGVNMCTLMAMDLGELCEDTITYNCPLLRQNEPEDIDCWCNSTSTWVTYGTCTTTGEHRRKKRSVALVPHVGMGLETRTETWMSSEGAWKHAQRIETWILRHPGFTIMAAILAYTIGTTHFQRALIFILLTAVAPSMT

SEQ ID NO: 114 - protein, 166 aa, Artificial Sequence
  DV3-prM-E89K
FHLTSRDGEPRMIVGKNERGKSLLFKTASGINMCTLIAMDLGEMCDDTVTYKCPHITEVEPEDIDCWCNLTSTWVTYGTCNQAGEHRRKKRSVALAPHVGMGLDTRTQTWMSAEGAWRQVEKVETWALRHPGFTILALFLAHYIGTSLTQKVVIFILLMLVTPSMT

SEQ ID NO: 115 - protein, 166 aa, Artificial Sequence
  DV4-prM-E89K
FHLSTRDGEPLMIVAKHERGRPLLFKTTEGINKCTLIAMDLGEMCEDTVTYKCPLLVNTEPEDIDCWCNLTSTWVMYGTCTQSGERRRKKRSVALTPHSGMGLETRAETWMSSEGAWKHAQRVESWILRNPGFALLAGFMAYMIGQTGIQRTVFFVLMMLVAPSYG

SEQ ID NO: 116 - protein, 166 aa, Artificial Sequence
  DV4-prM-E89N
FHLSTRDGEPLMIVAKHERGRPLLFKTTEGINKCTLIAMDLGEMCEDTVTYKCPLLVNTEPEDIDCWCNLTSTWVMYGTCTQSGERRRNKRSVALTPHSGMGLETRAETWMSSEGAWKHAQRVESWILRNPGFALLAGFMAYMIGQTGIQRTVFFVLMMLVAPSYG

SEQ ID NO: 117 - DNA, 26 bp, Artificial Sequence
  primer
gtaacttatgggacttgtactaccac

SEQ ID NO: 118 - DNA, 25 bp, Artificial Sequence
  primer
agcgcttatatctctgagcacgtcc

SEQ ID NO: 119 - DNA, 18 bp, Artificial Sequence
  primer
cacatgtggaacgagtgc

SEQ ID NO: 120 - DNA, 26 bp, Artificial Sequence
  primer
gtaacttatgggacttgtactaccac

SEQ ID NO: 121 - DNA, 24 bp, Artificial Sequence
  primer
cactgagcgcttacttctacgccc

SEQ ID NO: 122 - DNA, 18 bp, Artificial Sequence
  primer
cacatgtggaacgagtgc

SEQ ID NO: 123 - DNA, 22 bp, Artificial Sequence
  primer
tgggtaacttatgggacttgta

SEQ ID NO: 124 - DNA, 24 bp, Artificial Sequence
  primer
accacgggagaacatagaagagaa

SEQ ID NO: 125 - DNA, 18 bp, Artificial Sequence
  primer
cacatgtggaacgagtgc

SEQ ID NO: 126 - DNA, 20 bp, Artificial Sequence
  primer
caccataatggcagcaatcc

SEQ ID NO: 127 - DNA, 24 bp, Artificial Sequence
  primer
acgacacatttccagagagccctg

SEQ ID NO: 128 - DNA, 21 bp, Artificial Sequence
  primer
acagctgtcagtaagatgaaa

SEQ ID NO: 129 - DNA, 22 bp, Artificial Sequence
  primer
ctratytccatsccrtaccagc

SEQ ID NO: 130 - protein, 10 aa, Dengue virus type 1
TGEHRRDKRS

SEQ ID NO: 131 - protein, 10 aa, Dengue virus type 2
TGEHRREKRS

SEQ ID NO: 132 - protein, 10 aa, Dengue virus type 3
AGEHRRDKRS

SEQ ID NO: 133 - protein, 10 aa, Dengue virus type 4
SGERRREKRS

SEQ ID NO: 134 - protein, 10 aa, Unknown
  zika virus
KGEARRSRRA

SEQ ID NO: 135 - protein, 10 aa, West Nile virus
TRHSRRSRRS

SEQ ID NO: 136 - protein, 10 aa, Japanese encephalitis virus
TRHSKRSRRS

SEQ ID NO: 137 - protein, 10 aa, Yellow fever virus
AGRSRRSRRS

SEQ ID NO: 138 - protein, 10 aa, Tick-borne encephalitis virus
KQEGSRTRRS

SEQ ID NO: 139 - protein, 10 aa, Kyasanur forest disease virus
KPAGGRNRRS

SEQ ID NO: 140 - protein, 10 aa, Kunjin virus
TRHSRRSRRS

SEQ ID NO: 141 - protein, 10 aa, Murray Valley encephalitis virus
ARHSRRSRRS

SEQ ID NO: 142 - protein, 10 aa, St. Louis encephalitis virus
MGHSRRSRRS

SEQ ID NO: 143 - protein, 10 aa, Powassan virus
RQAGSRGKRS

SEQ ID NO: 144 - protein, 10 aa, Unknown
  Usutu virus
TRHSKRSRRS

SEQ ID NO: 145 - protein, 9 aa, Influenza A virus
QRERRKKRG

SEQ ID NO: 146 - protein, 10 aa, Unknown
  SARS-CoV-2
QTNSPRRARS

SEQ ID NO: 147 - protein, 10 aa, Artificial Sequence
  DV1 prM-D89K
TGEHRRKKRS

SEQ ID NO: 148 - protein, 10 aa, Artificial Sequence
  DV2 prM-E89K
TGEHRRKKRS

SEQ ID NO: 149 - protein, 10 aa, Artificial Sequence
  DV3 prM-D89K
AGEHRRKKRS

SEQ ID NO: 150 - protein, 10 aa, Artificial Sequence
  DV4 prM-E89K
SGERRRKKRS

SEQ ID NO: 151 - protein, 10 aa, Artificial Sequence
  DV4 prM-D89N
SGERRRNKRS

SEQ ID NO: 152 - protein, 10 aa, Artificial Sequence
  consensus sequence
  misc_feature (3)..(5): Xaa can be any naturally occurring amino acid
  misc_feature (7)..(7): Xaa can be any naturally occurring amino acid
TGXXXRXKRS

SEQ ID NO: 153 - protein, 7 aa, Artificial Sequence
  substituted DENV cleavage sequence
AMRRTKR

SEQ ID NO: 154 - protein, 7 aa, Artificial Sequence
  substituted DENV cleavage sequence
AARRSKR

SEQ ID NO: 155 - protein, 7 aa, Artificial Sequence
  substituted DENV cleavage sequence
HASRNKR

SEQ ID NO: 156 - protein, 7 aa, Artificial Sequence
  substituted DENV cleavage sequence
VSHRKKR

SEQ ID NO: 157 - protein, 7 aa, Artificial Sequence
  substituted DENV cleavage sequence
GNRRSKR

SEQ ID NO: 158 - protein, 7 aa, Artificial Sequence
  substituted DENV cleavage sequence
SKMRSKR

SEQ ID NO: 159 - protein, 7 aa, Artificial Sequence
  substituted DENV cleavage sequence
SRNRLKR

(19) United States
(12) Patent Application Publication
(10) Pub. No.: US 2024/0156941 A1
(43) Pub. Date: May 16, 2024

(54) METHODS AND COMPOSITIONS FOR MATURE DENGUE VIRUSES AS VACCINES AND DIAGNOSTICS

(71) Applicant: The University of North Carolina at Chapel Hill, Chapel Hill, NC (US)

(72) Inventors: Ralph Baric, Haw River, NC (US); Long Ping Victor Tse, Chapel Hill, NC (US); Rita Meganck, Chapel Hill, NC (US)

(21) Appl. No.: 18/550,730

(22) PCT Filed: Mar. 17, 2022
(86) PCT No.: PCT/US2022/020791

§ 371 Date: Sep. 15, 2023

Related U.S. Application Data
(60) Provisional application No. 63/162,063, filed on Mar. 17, 2021.

(51) Int. Cl. A61K 39/12; A61K 39/00; C07K 14/005; C12N 7/00; G01N 33/569

(52) U.S. Cl. CPC A61K 39/12; C07K 14/005; C12N 7/00; G01N 33/56983; A61K 2039/5256; A61K 2039/5258; A61K 2039/6075; C12N 2770/24122; C12N 2770/24123; C12N 2770/24134; C12N 2770/24151; G01N 2333/185; G01N 2469/20

(57) ABSTRACT

This invention relates to mature flavivirus particles and methods of making and using the same. This invention further relates to flavivirus prM glycoproteins, nucleic acids encoding the same, as well as particles, populations, and compositions comprising the same. Also disclosed are methods of making and using the prM glycoproteins of the invention.

### STATEMENT OF PRIORITY

This application claims the benefit, under 35 U.S.C. § 119(e), of U.S. Provisional Application Ser. No. 63/162,063, filed Mar. 17, 2021, the entire contents of which are incorporated by reference herein.

### STATEMENT REGARDING ELECTRONIC FILING OF A SEQUENCE LISTING

A Sequence Listing in ASCII text format, submitted under 37 C.F.R. § 1.821, entitled 5470-927-WO_ST25.txt, 47,108 bytes in size, generated on Mar. 17, 2022 and filed via EFS-Web, is provided in lieu of a paper copy. This Sequence Listing is hereby incorporated herein by reference into the specification for its disclosures FIELD OF THE INVENTION This invention relates to mature flavivirus particles and methods of making and using the same. This invention further relates to flavivirus prM glycoproteins, nucleic acids encoding the same, as well as particles, populations, and compositions comprising the same. Also disclosed are methods of making and using the prM glycoproteins of the invention.

### STATEMENT OF GOVERNMENT SUPPORT

This invention was made with government support under Grant Numbers AI107731, AI106695, and AI125198 awarded by the National Institutes of Health. The government has certain rights in the invention.

### BACKGROUND OF THE INVENTION

Mosquito-borne Dengue virus (DENV) is a major global public health threat causing ˜400 million new cases of dengue annually (Brady et al. 2012 PLoS Negl. Trop. Dis. 6; Bhatt et al. 2013 Nature 495:504-507). Although the majority of cases occur in tropical and subtropical areas where the mosquito vectors are most concentrated, global warming, travel, and globalization have contributed to the worldwide spread and intermixing of the four DENV serotypes (Messina et al. 2019 Nat. Microbiol. 4:1508-1515). A hallmark of DENV pathogenesis is the possibility for antibody dependent enhancement (ADE), which can progress to life threatening dengue hemorrhagic fever/dengue shock syndrome (DHF/DSS) upon secondary infection with a different serotype. No approved antiviral treatments are currently available to treat DENV disease and the only approved vaccine, Dengvaxia, is no longer recommended for use in naïve populations (Wilder-Smith et al. 2019 Lancet Infect. Dis. 19:e31-e38; Report, W.H.O. Dengue vaccine: WHO position paper, September 2018—Recommendations. Vaccine 37:4848-4849).

The present invention overcomes previous shortcomings in the art by providing recombinant flavivirus prM glycoproteins and mature flaviviruses with enhanced furin cleavability, antigenicity, and infectivity, e.g., as immunogens, and methods of using and making the same.

### SUMMARY OF THE INVENTION

One aspect of the present invention provides a recombinant flavivirus prM glycoprotein comprising a prM glycoprotein backbone and at least one amino acid substitution at position 89 (“P3”), 87 (“P5”), 86 (“P6”), and/or 85 in a furin cleavage site (e.g., positions 83-92), wherein the numbering corresponds to SEQ ID NO:104 [DENV2-prM], and wherein the at least one amino acid substitution introduces an amino acid residue in the furin cleavage site that is more basic than the original amino acid residue.

Also provided herein are isolated nucleic acid molecules (e.g., mRNA molecules, e.g., mRNA-LNP molecules for prophylactic and/or therapeutic immunogens), flavivirus particles or virus like particles (VLP), vectors, populations of flavivirus particles or VLP, and compositions comprising and/or encoding the flavivirus prM glycoprotein of the present invention.

In some embodiments, the particle of the invention may be in a mature form (e.g., may assemble in a herringbone structure formed from homodimers of E glycoproteins with icosahedral symmetry).

Another aspect of the invention provides a method of producing an immune response to a flavivirus in a subject, comprising administering to the subject an effective amount of the recombinant prM glycoprotein, flavivirus particle or VLP, nucleic acid molecule, vector, population, and/or composition of the present invention.

Another aspect of the invention provides a method of treating a flavivirus infection in a subject, comprising administering to the subject an effective amount of the recombinant prM glycoprotein, flavivirus particle or VLP, nucleic acid molecule, vector, population, and/or composition of the present invention.

Another aspect of the invention provides a method of preventing a disorder associated with flavivirus infection in a subject, comprising administering to the subject an effective amount of the recombinant prM glycoprotein, flavivirus particle or VLP, nucleic acid molecule, vector, population, and/or composition of the present invention.

Another aspect of the invention provides a method of protecting a subject from the effects of a flavivirus infection, comprising administering to the subject an effective amount of the recombinant prM glycoprotein, flavivirus particle or VLP, nucleic acid molecule, vector, population, and/or composition of the present invention.

Another aspect of the invention provides a method of identifying the presence of a maturation-dependent neutralizing antibody to a flavivirus in a biological sample from a subject, comprising: a) contacting a biological sample from the subject with the flavivirus particle or VLP of the invention, a composition comprising the same and/or an isolated nucleic acid encoding the same, in any combination, under conditions whereby neutralization of the flavivirus particles can be detected; and b) detecting neutralization in step (a), thereby identifying the presence of a maturation-dependent neutralizing antibody to the flavivirus in the biological sample from the subject.

Another aspect of the invention provides a method of identifying the presence of a maturation-dependent neutralizing antibody to a flavivirus in a biological sample from a subject, comprising: a) contacting a biological sample from a subject that has been administered an E glycoprotein of a flavivirus (e.g., an isolated E glycoprotein and/or a flavivirus comprising an E glycoprotein) with flavivirus particles comprising the recombinant prM glycoprotein of the present invention under conditions whereby neutralization of the flavivirus particles can be detected; and b) detecting neutralization in step (a), thereby identifying the presence of a maturation-dependent neutralizing antibody to the flavivirus in the biological sample from the subject.

Another aspect of the invention provides a method of detecting an antibody to a flavivirus in a sample, comprising: a) contacting the sample with the flavivirus particle or VLP of the present invention, a composition comprising the same and/or an isolated nucleic acid encoding the same, in any combination, under conditions whereby an antigen/antibody complex can form; and b) detecting formation of an antigen/antibody complex, thereby detecting an antibody to the flavivirus in the sample.

Another aspect of the invention provides a method of detecting an antibody to a flavivirus in a sample, comprising: a) contacting the sample with the recombinant prM glycoprotein of the present invention under conditions whereby an antigen/antibody complex can form; and b) detecting formation of an antigen/antibody complex, thereby detecting an antibody to the flavivirus in the sample.

Another aspect of the invention provides a method of producing a mature flavivirus particle, comprising: constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); thereby producing a mature flavivirus particle.

Another aspect of the invention provides a method of increasing production of a mature form of a flavivirus particle, comprising: constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); thereby increasing production of the mature form of the flavivirus particle.

Another aspect of the invention provides a method of enhancing antigenicity of a flavivirus particle, comprising: constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); thereby enhancing the antigenicity of the flavivirus particle.

Another aspect of the invention provides a method of enhancing infectivity of a flavivirus particle, comprising: constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); thereby enhancing the infectivity of the flavivirus particle.

Another aspect of the invention provides a method of enhancing antigenicity of a population of flavivirus particles, comprising: (a) constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); and (b) producing a population of the flavivirus particle of (a) (e.g., culturing the flavivirus particle, e.g., culturing in insect cell cultures and/or mammalian cell cultures); thereby enhancing the antigenicity of the population of flavivirus particles.

Another aspect of the invention provides a method of enhancing infectivity of a population of flavivirus particles, (a) constructing a flavivirus particle comprising the substituted prM glycoprotein of the present invention, wherein the presence of the substituted prM glycoprotein induces the flavivirus particle to assemble in a mature form (e.g., assemble in a herringbone structure formed from homodimers of E glycoproteins with icosahedral symmetry); and (b) producing a population of the flavivirus particle of (b) (e.g., culturing the flavivirus particle, e.g., culturing in insect cell cultures and/or mammalian cell cultures); thereby enhancing the infectivity of the population of flavivirus particles.

In some embodiments, the substituted prM glycoprotein of the present invention may be substituted via mutagenesis, including but not limited to, site-directed mutagenesis, saturation mutagenesis, directed evolution, deep mutagenesis, unnatural amino acid incorporation, post-translational modification, or any combination thereof.

### BRIEF DESCRIPTION OF THE DRAWINGS

FIGS. 1A-1B: Furin cleavage site alignment and DENV maturation. (FIG. 1A) Amino acid sequence alignment of viral furin cleavage sites from position 9 (P9) to position 1 prime (P1′). Pi-Tou scores are the prediction of logarithmic-odd probabilities of all the different viral furin cleavage sites (higher value=better substrate for furin). DENV1, 2, 3 and 4 (SEQ ID NO:130-133), Zika virus (ZIKV; SEQ ID NO:134), West Nile virus (WNV; SEQ ID NO:135), Japanese encephalitis virus (JEV; SEQ ID NO:136), yellow fever virus (YFV; SEQ ID NO:137), Tick-borne encephalitis virus (TBEV; SEQ ID NO:138), Kyasanur Forest disease virus (KFDV; SEQ ID NO:139), Kunjin virus (KUN; SEQ ID NO:140), Murray Valley encephalitis virus (MVEV; SEQ ID NO:141), St. Louis encephalitis virus (SLEV; SEQ ID NO:142), Powassan virus (POW; SEQ ID NO:143), Usutu virus (USUV; SEQ ID NO:144), highly pathogenic avian influenza virus (HPAI; SEQ ID NO:145) and SARS-Coronavirus-2 (SEQ ID NO:146). (FIG. 1B) Representative western blot image of DV1-WT, DV2-WT, DV3-WT, and DV4-WT viral supernatants grown in either Vero or C6/36 blotted with anti-Env and anti-prM antibodies.

FIG. 2: Growth kinetics and maturation status of DENVs on Vero and VF1 cells. (FIG. 2, panel A) Schematic of the Sleeping Beauty-based transposon cassette for ectopic expression of human furin (hFurin). A bi-directional EF1a promoter was used to drive the expression of hFurin and red-fluorescent protein (RFP) with a puromycin resistance gene (Puro) linked by a 2A self-cleaving peptide (P2A). (FIG. 2, panel B) Western blot and (FIG. 2, panel C) immunofluorescence images of polyclonal and clonal VF1 using anti-furin antibodies. Growth kinetics and degree of maturation of (FIG. 2, panel D) DENV1-WT, (FIG. 2, panel E) DENV2-WT, (FIG. 2, panel F) DENV3-WT and (FIG. 2, panel G) DENV4-WT in unmodified Vero cells (Circle) and VF1 cells (Triangle). Cells were infected with DENV at MOI 0.01-0.05 for 120 hours. Supernatants were harvested every 24 h for 120 h and the 120 h supernatants were analyzed by western blot for DENV maturation using anti-Env and anti-prM antibodies. All assays were performed with at least two biological repeats with two technical replicates. Growth kinetics of DENV variants were compared to their corresponding wildtype using 2-way ANOVA multiple comparisons.

FIG. 3: Generation of mature DENV1 and DENV2 via genetic modification. (FIG. 3, panel A) Sequence composition of prM cleavage sites of DV1-WT and DV1-prM-D89K (SEQ ID NO:130 and 147). (FIG. 3, panel B) Growth kinetics of DV1-WT and DV1-prM-D89K in Vero and C6/36 cells. (FIG. 3, panel C) Representative western blot image (bottom) of DV1-WT and DV1-prM-D89K viral supernatants blotted with anti-Env and anti-prM antibodies, and quantification (top) of viral maturation (prM/Env) normalized to DV1-WT (lower value=more mature). (FIG. 3, panel D) Sequence composition of prM cleavage sites of DV2-WT and DV2-prM-E89K (SEQ ID NO:131 and 148). (FIG. 3, panel E) Growth kinetics of DV2-WT and DV2-prM-E89K in Vero and C6/36 cells. (FIG. 3, panel F) Representative western blot image (bottom) of DV2-WT and DV2-prM-E89K viral supernatants blotted with anti-Env and anti-prM antibodies, and quantification (top) of viral maturation normalized to DV2-WT. Growth kinetics and maturation of DENV variants were compared to their corresponding wildtype virus using 2-way ANOVA multiple comparisons.

FIG. 4: Iterative genetic optimization of mature DENV3 and DENV4. (FIG. 4, panel A) Sequence composition of prM cleavage sites of DV3-WT and DV3-prM-D89K (SEQ ID NO:132 and 149). (FIG. 4, panel B) Growth kinetics of DV3-WT and DV3-prM-D89K in C6/36 and Vero cells. (FIG. 4, panel C) Representative western blot image (bottom) of DV3-WT and DV3-prM-D89K viral supernatants blotted with anti-Env and anti-prM antibodies and quantification (top) of viral maturation (prM/Env) normalized to DV3-WT. All titers were determined on C6/36 cells. (FIG. 4, panel D) Sequence composition of prM cleavage sites of DV4-WT, DV4-prM-E89K, and DV4-prM-E89N (SEQ ID NO:133, 150 and 151). Growth kinetics of DV4-WT, DV4-prM-E89K, and DV4-prM-E89N in (FIG. 4, panel E) C6/36 and (FIG. 4, panel F) Vero cells. (FIG. 4, panel G) Representative western blot image (bottom) of DV4-WT, DV4-prM-E89K, and DV4-prM-E89N viral supernatants blotted with anti-Env and anti-prM antibodies and quantification (top) of viral maturation (prM/Env) normalized to DV4-WT. Growth kinetics and maturation of DENV variants were compared to their corresponding wildtype using 2-way ANOVA multiple comparisons.

FIG. 5: Directed-evolution of DENV2 prM cleavage site in Vero and C6/36 cells. (FIG. 5, panel A) Schematic of directed-evolution from library generation to high-throughput sequencing. Enrichment plots of prM cleavage site sequences from plasmid library to viral population at the 3^{rd} passage (P3) and the proportion as well as sequence of the Top 5 enriched sequences in (FIG. 5, panel B) C6/36 and (FIG. 5, panel C) Vero cells. Sequences shown include consensus SEQ ID NO:152 (FIG. 5, panel A), and RAQRYKR (residues 3-9 of SEQ ID NO:103; FIG. 5, panel B), AMRRTKR (SEQ ID NO:153, FIG. 5 panel B), AARRSKR (SEQ ID NO:154, FIG. 5 panel B), HASRNKR (SEQ ID NO:155, FIG. 5 panel B), VSHRKKR (SEQ ID NO:156, FIG. 5 panel B), AGRRSKR (residues 3-9 of SEQ ID NO:101; FIG. 5, panel C), GNRRSKR (SEQ ID NO:157, FIB. 5 panel C), SKMRSKR (SEQ ID NO:158, FIG. 5 panel C), SRNRLKR (SEQ ID NO:159, FIG. 5 panel C).

FIG. 6: Replicative fitness and maturation of evolved DV2 variants. Growth kinetics of DV2-WT, DV2-C1, and DV2-V1 in (FIG. 6, panel A) Vero and (FIG. 6, panel B) C6/36 cells. (FIG. 6, panel C) Sequence composition of prM cleavage sites of DV2-WT (SEQ ID NO:131), DV2-C1 (SEQ ID NO:103) and DV2-V1 (SEQ ID NO:101). (FIG. 6, panel D) Representative western blot image (bottom) of DV2-WT, DV2-C1, and DV2-V1 viral supernatants blotted with anti-Env and anti-prM antibodies, and quantification (top) of viral maturation (prM/Env) normalized to DV2-WT (lower value=more mature). (FIG. 6, panel E) Competition assay of DV2-WT, DV2-C1, and DV2-V1 at equal FFU ratio in Vero and C6/36 cells at high and low MOI. Viral genomes were quantified by digital droplet PCR (ddPCR) multiplexed with a general DV2 probe and strain specific probes. Growth kinetics and maturation of DV2 variants were compared to DV2-WT using 2-way ANOVA multiple comparisons.

FIG. 7: Antigenic profile of mature DENV. (FIG. 7, panel A) FRNT₅₀ of DV1-WT and DV1-prM-D89K against 1C19, EDE1-C10 (C10) and 1M7. (FIG. 7, panel B) FRNT₅₀ of DV4-WT and DV4-prM-E89N against D4-126, D4-131, C10 and 1M7. (FIG. 7, panel C) FRNT₅₀ of DV2-WT and DV2-V1 against 1C19, EDE2-B7 (B7), C10 and 1M7. (FIG. 7, panel D) FRNT₅₀ of vaccine sera and traveler serum against DV4 and DV4-prM-E89N.

FIG. 8: (top) DV4 growth kinetics at 37° C. for indicated viruses; (bottom) DV4 growth kinetics at 32° C. for indicated viruses.

FIG. 9 shows blot data identifying the E, prM, C, and M proteins of DV4-WT and DV4-prM-E89N grown in Vero cell culture (left) and VF1 cell culture (right).

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the term “nucleic acid” encompasses both RNA and DNA, including cDNA, genomic DNA, synthetic (e.g., chemically synthesized) DNA and chimeras of RNA and DNA. The nucleic acid may be double-stranded or single-stranded. The nucleic acid may be synthesized using nucleotide analogs or derivatives (e.g., inosine or phosphorothioate nucleotides). Such nucleotides can be used, for example, to prepare nucleic acids that have altered base-pairing abilities or increased resistance to nucleases.

The terms “nucleic acid segment,” “nucleotide sequence,” “nucleic acid molecule,” or more generally “segment” will be understood by those in the art as a functional term that includes both genomic DNA sequences, ribosomal RNA sequences, transfer RNA sequences, messenger RNA sequences, small regulatory RNAs, operon sequences and smaller engineered nucleotide sequences that express or may be adapted to express, proteins, polypeptides or peptides. Nucleic acids of the present disclosure may also be synthesized, either completely or in part, by methods known in the art. Thus, all or a portion of the nucleic acids of the present codons may be synthesized using codons preferred by a selected host. Species-preferred codons may be determined, for example, from the codons used most frequently in the proteins expressed in a particular host species. Other modifications of the nucleotide sequences may result in mutants having slightly altered activity.

The term “sequence identity,” as used herein, has the standard meaning in the art. As is known in the art, a number of different programs can be used to identify whether a polynucleotide or polypeptide has sequence identity or similarity to a known sequence.

Sequence identity or similarity may be determined using standard techniques known in the art, including, but not limited to, the local sequence identity algorithm of Smith & Waterman, Adv. Appl. Math. 2:482 (1981), by the sequence identity alignment algorithm of Needleman & Wunsch, J Mol. Biol. 48:443 (1970), by the search for similarity method of Pearson & Lipman, Proc. Natl. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12:387 (1984), preferably using the default settings, or by inspection.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351 (1987); the method is similar to that described by Higgins & Sharp, CABOS 5:151 (1989).

Another example of a useful algorithm is the BLAST algorithm, described in Altschul et al., J. Mol. Biol. 215:403 (1990) and Karlin et al., Proc. Natl. Acad. Sci. USA 90:5873 (1993). A particularly useful BLAST program is the WU-BLAST-2 program which was obtained from Altschul et al., Meth. Enzymol. 266:460 (1996);
blast.wustl/edu/blast/README.html. WU-BLAST-2 uses several search parameters, which are preferably set to the default values. The parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucleic Acids Res. 25:3389 (1997).

A percentage amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, percent nucleic acid sequence identity is defined as the percentage of nucleotide residues in the candidate sequence that are identical with the nucleotides in the polynucleotide specifically disclosed herein.

The alignment may include the introduction of gaps in the sequences to be aligned. In addition, for sequences which contain either more or fewer nucleotides than the polynucleotides specifically disclosed herein, it is understood that in one embodiment, the percentage of sequence identity will be determined based on the number of identical nucleotides in relation to the total number of nucleotides. Thus, for example, sequence identity of sequences shorter than a sequence specifically disclosed herein, will be determined using the number of nucleotides in the shorter sequence, in one embodiment. In percent identity calculations relative weight is not assigned to various manifestations of sequence variation, such as insertions, deletions, substitutions, etc.

In one embodiment, only identities are scored positively (+1) and all forms of sequence variation including gaps are assigned a value of “0,” which obviates the need for a weighted scale or parameters as described below for sequence similarity calculations. Percent sequence identity can be calculated, for example, by dividing the number of matching identical residues by the total number of residues of the “shorter” sequence in the aligned region and multiplying by 100. The “longer” sequence is the one having the most actual residues in the aligned region.

As used herein, the term “polypeptide” encompasses both peptides and proteins (including fusion proteins), unless indicated otherwise.

As used herein, the term “chimera,” “chimeric,” and/or “fusion protein” refer to an amino acid sequence (e.g., polypeptide) generated non-naturally by deliberate human design comprising, among other components, an amino acid sequence of a protein of interest and/or a modified variant and/or active fragment thereof (a “backbone”), wherein the protein of interest comprises modifications (e.g., substitutions such as singular residues and/or contiguous regions of amino acid residues) from different wild type reference sequences (chimera), optionally linked to other amino acid segments (fusion protein). The different components of the designed protein may provide differing and/or combinatorial function. Structural and functional components of the designed protein may be incorporated from differing and/or a plurality of source material. The designed protein may be delivered exogenously to a subject, wherein it would be exogenous in comparison to a corresponding endogenous protein.

As used herein with respect to nucleic acids, the term “operably linked” refers to a functional linkage between two or more nucleic acids. For example, a promoter sequence may be described as being “operably linked” to a heterologous nucleic acid sequence because the promoter sequence initiates and/or mediates transcription of the heterologous nucleic acid sequence. In some embodiments, the operably linked nucleic acid sequences are contiguous and/or are in the same reading frame.

In embodiments of the invention, an “immunogenically active fragment” of a flavivirus polypeptide (e.g., the E protein) comprises, consists essentially of or consists of at least about 6, 8, 10, 12, 15, 20, 30, 50, 75, 100, 125, 150, 200, 250, 300, 350, 400, 450 or more amino acids, optionally contiguous amino acids, and/or less than about 495, 475, 450, 425, 400, 350, 300, 250, 200, 150, 100, 75 or 50 amino acids, optionally contiguous amino acids, including any combination of the foregoing as long as the lower limit is less than the upper limit, and the “immunogenically active fragment” induces an immune response (e.g., IgG and/or IgA that react with the native antigen), optionally a protective immune response, against dengue virus in a host and induces the production of antibodies that specifically bind to the quaternary dengue virus epitope newly identified by the inventors.

The term “epitope” as used herein means a specific amino acid sequence that, when present in the proper conformation, provides a reactive site for an antibody (e.g., B cell epitope) or T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715.

Similarly, conformational epitopes can be readily identified by determining spatial conformation of amino acids such as by, e.g., x-ray crystallography and 2-dimensional nuclear magnetic resonance. Antigenic regions of proteins can also be identified using standard antigenicity and hydropathy plots, such as those calculated using, e.g., the Omiga version 1.0 software program available from the Oxford Molecular Group. This computer program employs the Hopp/Woods method (Hopp et al. Proc. Natl. Acad. Sci USA (1981) 78:3824-3828) for determining antigenicity profiles and the Kyte-Doolittle technique (Kyte et al. J. Mol. Biol. (1982) 157:105-132) for hydropathy plots.

Generally, T-cell epitopes that are involved in stimulating the cellular arm of a subject's immune system are short peptides of about 8-25 amino acids. A common way to identify T-cell epitopes is to use overlapping synthetic peptides and analyze pools of these peptides, or the individual ones, that are recognized by T cells from animals that are immune to the antigen of interest, using, for example, an enzyme-linked immunospot assay (ELISPOT). These overlapping peptides can also be used in other assays such as the stimulation of cytokine release or secretion, or evaluated by constructing major histocompatibility (MHC) tetramers containing the peptide. Such immunogenically active fragments can also be identified based on their ability to stimulate lymphocyte proliferation in response to stimulation by various fragments from the antigen of interest.

A “recombinant” nucleic acid, polynucleotide or nucleotide sequence is one produced by genetic engineering techniques.

A “recombinant” polypeptide is produced from a recombinant nucleic acid, polypeptide or nucleotide sequence.

As used herein, an “isolated” polynucleotide (e.g., an “isolated nucleic acid” or an “isolated nucleotide sequence”) means a polynucleotide at least partially separated from at least some of the other components of the naturally occurring organism or virus, for example, the cell or viral structural components or other polypeptides or nucleic acids commonly found associated with the polynucleotide. Optionally, but not necessarily, the “isolated” polynucleotide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polynucleotide is at least about 1%, 5%, 10%, 20%, 30%, 40%, 50%, 60%, 70%, 80%, 90%, 95% or more pure.

An “isolated” polypeptide means a polypeptide that is at least partially separated from at least some of the other components of the naturally occurring organism or virus, for example, the cell or viral structural components or other polypeptides or nucleic acids commonly found associated with the polypeptide. Optionally, but not necessarily, the “isolated” polypeptide is present at a greater concentration (i.e., is enriched) as compared with the starting material (e.g., at least about a two-fold, three-fold, four-fold, ten-fold, twenty-fold, fifty-fold, one-hundred-fold, five-hundred-fold, one thousand-fold, ten thousand-fold or greater concentration). In representative embodiments, the isolated polypeptide is at least about 1%, 5%, 10%, 20%, 30%, 40%, 50%, 60%, 70%, 80%, 90%, 95% or more pure.

Furthermore, an “isolated” cell is a cell that has been partially or completely separated from other components with which it is normally associated in nature. For example, an isolated cell can be a cell in culture medium and/or a cell in a pharmaceutically acceptable carrier.

The term “endogenous” refers to a component naturally found in an environment, i.e., a gene, nucleic acid, miRNA, protein, cell, or other natural component expressed in the subject, as distinguished from an introduced component, i.e., an “exogenous” component.

As used herein, the term “heterologous” refers to a nucleotide/polypeptide that originates from a foreign species, or, if from the same species, is substantially modified from its native form in composition and/or genomic locus by deliberate human intervention.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive amino acids.

As used herein with respect to nucleic acids, the term “functional fragment” or “active fragment” refers to nucleic acid that encodes a functional fragment of a polypeptide.

As used herein with respect to polypeptides, the term “functional fragment” or “active fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., the ability to up- or down-regulate gene expression). In some embodiments, the functional fragment actually has a higher level of at least one biological activity of the full-length polypeptide.

As used herein, the term “modified,” as applied to a polynucleotide or polypeptide sequence, refers to a sequence that differs from a wild-type sequence due to one or more deletions, additions, substitutions, or any combination thereof. Modified sequences may also be referred to as “modified variant(s).”

The terms “immunogen” and “antigen” are used interchangeably herein and mean any compound (including polypeptides) to which a cellular and/or humoral immune response can be directed. In particular embodiments, an immunogen or antigen can induce a protective immune response against the effects of dengue virus infection.

A “vector” refers to a compound used as a vehicle to carry foreign genetic material into another cell, where it can be replicated and/or expressed. A cloning vector containing foreign nucleic acid is termed a recombinant vector. Examples of nucleic acid vectors are plasmids, viral vectors, cosmids, expression cassettes, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs or expression cassettes) are for the expression of the exogenous gene in the target cell, and generally have a promoter sequence that drives expression of the exogenous gene. Insertion of a vector into the target cell is referred to transformation or transfection for bacterial and eukaryotic cells, although insertion of a viral vector is often called transduction. The term “vector” may also be used in general to describe items to that serve to carry foreign genetic material into another cell, such as, but not limited to, a transformed cell or a nanoparticle.

As used herein, by “isolate” or “purify” (or grammatical equivalents) a vector, it is meant that the vector is at least partially separated from at least some of the other components in the starting material.

“Effective amount” as used herein refers to an amount of a vector, nucleic acid, epitope, polypeptide, cell, particle, VLP, composition or formulation of the invention that is sufficient to produce a desired effect, which can be a therapeutic and/or beneficial effect. The effective amount will vary with the age, general condition of the subject, the severity of the condition being treated, the particular agent administered, the duration of the treatment, the nature of any concurrent treatment, the pharmaceutically acceptable carrier used, and like factors within the knowledge and expertise of those skilled in the art. As appropriate, an “effective amount” in any individual case can be determined by one of ordinary skill in the art by reference to the pertinent texts and literature and/or by using routine experimentation.

The term “immunogenic amount” or “effective immunizing dose,” as used herein, unless otherwise indicated, means an amount or dose sufficient to induce an immune response (which can optionally be a protective response) in the treated subject that is greater than the inherent immunity of non-immunized subjects. An immunogenic amount or effective immunizing dose in any particular context can be routinely determined using methods known in the art.

The terms “vaccine,” “vaccination” and “immunization” are well-understood in the art, and are used interchangeably herein. For example, the terms vaccine, vaccination or immunization can be understood to be a process or composition that increases a subject's immune reaction to an immunogen (e.g., by providing an active immune response), and therefore its ability to resist, overcome and/or recover from infection (i.e., a protective immune response).

By the terms “treat,” “treating” or “treatment of” (and grammatical variations thereof) it is meant that the severity of the subject's condition is reduced, at least partially improved or ameliorated and/or that some alleviation, mitigation or decrease in at least one clinical symptom is achieved and/or there is a delay in the progression of the disease or disorder. In representative embodiments, the terms “treat,” “treating” or “treatment of” (and grammatical variations thereof) refer to a reduction in the severity of viremia and/or a delay in the progression of viremia, with or without other signs of clinical disease.

A “treatment effective” amount as used herein is an amount that is sufficient to treat (as defined herein) the subject. Those skilled in the art will appreciate that the therapeutic effects need not be complete or curative, as long as some benefit is provided to the subject.

The term “prevent,” “preventing” or “prevention of” (and grammatical variations thereof) refer to prevention and/or delay of the onset and/or progression of a disease, disorder and/or a clinical symptom(s) in a subject and/or a reduction in the severity of the onset and/or progression of the disease, disorder and/or clinical symptom(s) relative to what would occur in the absence of the methods of the invention. In representative embodiments, the terms “prevent,” “preventing” or “prevention of” (and grammatical variations thereof) refer to prevention and/or delay of the onset and/or progression of viremia in the subject, with or without other signs of clinical disease. The prevention can be complete, e.g., the total absence of the disease, disorder and/or clinical symptom(s). The prevention can also be partial, such that the occurrence of the disease, disorder and/or clinical symptom(s) in the subject and/or the severity of onset and/or the progression is less than what would occur in the absence of the present invention.

A “prevention effective” amount as used herein is an amount that is sufficient to prevent (as defined herein) the disease, disorder and/or clinical symptom in the subject. Those skilled in the art will appreciate that the level of prevention need not be complete, as long as some benefit is provided to the subject.

The efficacy of treating and/or preventing dengue virus infection by the methods of the present invention can be determined by detecting a clinical improvement as indicated by a change in the subject's symptoms and/or clinical parameters (e.g., viremia), as would be well known to one of skill in the art.

Unless indicated otherwise, the terms “protect,” “protecting,” “protection” and “protective” (and grammatical variations thereof) encompass both methods of preventing and treating dengue virus infection in a subject, whether against one or multiple strains, genotypes or serotypes of dengue virus.

The terms “protective” immune response or “protective” immunity as used herein indicates that the immune response confers some benefit to the subject in that it prevents or reduces the incidence and/or severity and/or duration of disease or any other manifestation of infection. For example, in representative embodiments, a protective immune response or protective immunity results in reduced viremia, whether or not accompanied by clinical disease. Alternatively, a protective immune response or protective immunity may be useful in the therapeutic treatment of existing disease.

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to immunogens by infection or by vaccination. Active immunity can be contrasted with passive immunity, which is acquired through the “transfer of preformed substances (antibody, transfer factor, thymic graft, interleukin-2) from an actively immunized host to a non-immune host.” Id.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold, and/or at least about 5%, 10%, 15%, 20%, 25%, 30%, 35%, 40%, 45%, 50%, 55%, 60%, 65%, 70%, 75%, 80%, 85%, 90%, 95%, or 100% or more, or any value or range therein.

The term “inhibit” or “reduce” or grammatical variations thereof as used herein refers to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

As used herein, “expression” refers to the process by which a polynucleotide is transcribed from a DNA template (such as into an mRNA or other RNA transcript) and/or the process by which a transcribed mRNA is subsequently translated into peptides, polypeptides, or proteins. Transcripts may be referred to as “transcription products” and encoded polypeptides may be referred to as “translation products.” Transcripts and encoded polypeptides may be collectively referred to as “gene products.” If the polynucleotide is derived from genomic DNA, expression may include splicing of the mRNA in a eukaryotic cell. The expression product itself, e.g., the resulting nucleic acid or protein, may also be said to be “expressed.” An expression product can be characterized as intracellular, extracellular, or secreted. The term “intracellular” means something that is inside a cell. The term “extracellular” means something that is outside a cell. A substance is “secreted” by a cell if it appears in significant measure outside the cell, from somewhere on or inside the cell.

A “subject” of the invention includes any animal susceptible to dengue virus infection. Such a subject is generally a mammalian subject (e.g., a laboratory animal such as a rat, mouse, guinea pig, rabbit, primates, etc.), a farm or commercial animal (e.g., a cow, horse, goat, donkey, sheep, etc.), or a domestic animal (e.g., cat, dog, ferret, etc.). In particular embodiments, the subject is a primate subject, a non-human primate subject (e.g., a chimpanzee, baboon, monkey, gorilla, etc.) or a human. Subjects of the invention can be a subject known or believed to be at risk of infection by a flavivirus (e.g., dengue virus). Alternatively, a subject according to the invention can also include a subject not previously known or suspected to be infected by a flavivirus or in need of treatment for flavivirus infection.

Subjects may be treated for any purpose, such as for eliciting a protective immune response or for eliciting the production of antibodies in that subject, which antibodies can be collected and used for other purposes such as research or diagnostic purposes or for administering to other subjects to produce passive immunity therein, etc.

Subjects include males and/or females of any age, including neonates, juvenile, mature and geriatric subjects. With respect to human subjects, in representative embodiments, the subject can be an infant (e.g., less than about 12 months, 10 months, 9 months, 8 months, 7 months, 6 months, or younger), a toddler (e.g., at least about 12, 18 or 24 months and/or less than about 36, 30 or 24 months), or a child (e.g., at least about 1, 2, 3, 4 or 5 years of age and/or less than about 14, 12, 10, 8, 7, 6, 5, or 4 years of age). In embodiments of the invention, the subject is a human subject that is from about 0 to 3, 4, 5, 6, 9, 12, 15, 18, 24, 30, 36, 48 or 60 months of age, from about 3 to 6, 9, 12, 15, 18, 24, 30, 36, 48 or 60 months of age, from about 6 to 9, 12, 15, 18, 24, 30, 36, 48 or 60 months of age, from about 9 to 12, 15, 18, 24, 30, 36, 48 or 60 months of age, from about 12 to 18, 24, 36, 48 or 60 months of age, from about 18 to 24, 30, 36, 48 or 60 months of age, or from about 24 to 30, 36, 48 or 60 months of age.

A “subject in need” of the methods of the invention can be a subject known to be, or suspected of being, infected with, or at risk of being infected with, a flavivirus.

The term “administering” or “administration” of a composition of the present invention to a subject includes any route of introducing or delivering to a subject a compound to perform its intended function (e.g., for use as a vaccine antigen). Administration includes self-administration and the administration by another.

A “sample” or “biological sample” of this invention can be any biological material, such as a biological fluid, an extract from a cell, an extracellular matrix isolated from a cell, a cell (in solution or bound to a solid support), a tissue, a tissue homogenate, and the like as are well known in the art.

### Compositions

Maturation of Dengue viruses (DENV) alters the structure, immunity and infectivity of the virion, and mature particles represent the dominant form in vivo. The production of highly mature virions principally relies on the structure and function of the viral premature membrane protein (prM) and its cleavage by the host protease furin. Using protein engineering and directed evolution of the prM cleavage site, the inventors of the present invention engineered genetically stable mature DENV viruses in all serotypes independent of cell or host, and demonstrated that the resulting mature DENV viruses were antigenically distinct from their isogenic partially mature forms.

The infectious, mature DENV composed of 90 E homodimers lying flat in a “herringbone” structure and organized into a 50 nm icosahedral (T=pseudo 3) symmetry (Kuhn et al. 2002 Cell 108:717-725). In contrast, the non-infectious immature DENV adopts a completely distinct structure as a 60 nm “spikey” sphere with 60 three-fold spikes (Zybert et al. 2008 J. Gen. Virol. 89:3047-3051; Plevka et al. 2014 J. Struct. Biol. 185:27-31; Kostyuchenko et al. 2013 J. Virol. 87:7700-7707). A third form of DENV is the partially mature/immature particles which adopt both mature and immature structure in different regions which generate limitless different topologies in the viral population.

While not wishing to be bound to theory, the maturation process is thought to provide evolutionary advantages in virus infection, immunity, and antigenic variation, although the biological functions of these interchanging maturation forms remain unknown. While the maturation status of common laboratory DENV strains varies, clinical isolates are typically more mature. Further description of the maturation status of dengue virus may be found in Pierson and Diamond, 2012 Curr. Opin. Virol. 2:168-175, and Galula et al. 2019 Hum. Vaccines Immunother. 15:2328-2336, incorporated herein by reference.

Proteolytic cleavage of viral membrane fusion proteins is a strategy for temporal or spatial control of virus infection, ultimately affecting tropism and transmission (White et al. 2008 Crit. Rev. Biochem. Mol. Biol. 43:189-219). In DENV, maturation is controlled by furin cleavage of the prM. Furin is a ubiquitously expressed host serine protease in the trans-Golgi network (TGN). Cleavage of prM releases the pr portion from the virion, and triggers a rotation and collapse of E protein to form the mature virion. In addition to conformational change in proteins, lipids may also play a role in stabilizing the mature DENV structure.

One aspect of the present invention provides a recombinant flavivirus prM glycoprotein comprising a prM glycoprotein backbone and at least one amino acid substitution at position 89 (“P3”), 87 (“P5”), 86 (“P6”), and/or 85 in a furin cleavage site (e.g., positions 83-92), wherein the numbering corresponds to SEQ ID NO:104 [DENV2-prM], and wherein the at least one amino acid substitution introduces an amino acid residue in the furin cleavage site that is more basic than the original amino acid residue.

In some embodiments, the furin cleavage site comprises an amino acid substitution at position 89. In some embodiments, the furin cleavage site comprises an amino acid substitution at position 89 and further amino acid substitutions at positions 87, 86, and/or 85.

In some embodiments, the furin cleavage site comprises an amino acid substitution at positions 87, 86, and/or 85, and no amino acid substitution at position 87. In some embodiments, the at least one amino acid substitution introduces an amino acid residue in the furin cleavage site that is more basic than the original amino acid residue may be any amino acid. As used herein, the term “amino acid” or “amino acid residue” encompasses any naturally occurring amino acid, modified forms thereof, and synthetic amino acids.

Naturally occurring, levorotatory (L-) amino acids as shown in Table 6.

Conservative amino acid substitutions are known in the art. In particular embodiments, a conservative amino acid substitution includes substitutions within one or more of the following groups: glycine, alanine; valine, isoleucine, leucine; aspartic acid, glutamic acid; asparagine, glutamine; serine, threonine; lysine, arginine; and/or phenylalanine, tyrosine.

Alternatively, the amino acid can be a modified amino acid residue (nonlimiting examples are shown in Table 7) and/or can be an amino acid that is modified by post-translation modification (e.g., acetylation, amidation, formylation, hydroxylation, methylation, phosphorylation or sulfatation). In addition, the non-naturally occurring amino acid can be an “unnatural” amino acid such as described by Wang et al., Annu Rev Biophys Biomol Struct. 35:225-49 (2006)). These unnatural amino acids can advantageously be used to chemically link molecules of interest to a virus particle.

In some embodiments, an amino acid residue is substituted adjacent to an insertion. In some embodiments, a substitution may comprise a deletion of one or more amino acid residues. The substitution(s) and residue positions may be described in one or more ways that are redundant in generating the same resultant amino acid sequence. Thus, a disclosure of one such description is herein considered a disclosure of each and inclusive of all such redundant disclosures. Thus, wherein the resultant amino acid sequence is identical, any disclosure provided herein describing one way to produce the resultant amino acid sequence is considered a disclosure of each and inclusive of all such redundant disclosures.

In some embodiments, the amino acid substitution comprises a substitution of the amino acid residue with a lysine (K), histidine (H), arginine (R), tyrosine (Y), serine (S), glutamic acid (E), valine (V), glycine (G), aspartic acid (D), methionine (M), glutamine (Q), asparagine (N), alanine (A), isoleucine (I), proline (P), phenylalanine (F), threonine (T), leucine (L), tryptophan (W), cysteine (C), or any derivative thereof at amino acid position 89 (i.e., 89K, 89H, 89R, etc.) of the backbone prM glycoprotein.

In some embodiments, the substitution of an amino acid residue that is more basic comprises 89K (e.g., E89K, D89K). In some embodiments, the substitution of an amino acid residue that is more basic comprises 89N (e.g., E89N, D89N). In some embodiments, the substitution of an amino acid residue that is more basic comprises 89Y (e.g., E89Y, D89Y). In some embodiments, the substitution of an amino acid residue that is more basic comprises 89S (e.g., E89S, D89S).

In some embodiments, the one or more amino acid substitutions at positions 87, 86, and 85 comprise 85R, 85A, 85S, 86A, 86G, 86K, 87Q, 87R, 87M, or any combination thereof.

In some embodiments, the furin cleavage site has enhanced cleavability by a furin enzyme, e.g., as compared to an unsubstituted furin cleavage site in a corresponding wildtype flavivirus prM glycoprotein.

In some embodiments, the furin cleavage site cleavability is about 1.5-fold enhanced, e.g., about 1.5-, 2-, 2.5-, 3-, 3.5-, 4-, 4.5-, 5-, 5.5-, 6-, 6.5-, 7-, 7.5-, 8-, 8.5-, 9-, 9.5-, or 10-fold enhanced or higher, or any value or range therein. Enhancement of furin cleavage site cleavability may be measured by any standard method in the art, as will be understood by the skilled artisan upon review of the disclosures of the present invention. Non-limiting examples include use of Pi-Tou score and/or a ProP score, as described in Tian et al. 2012 Sci. Rep. 2:261, incorporated herein by reference in its entirety. For example, in some embodiments, the furin cleavage site cleavability is about 1.5-fold enhanced, e.g., about 1.5-, 2-, 2.5-, 3-, 3.5-, 4-, 4.5-, 5-, 5.5-, 6-, 6.5-, 7-, 7.5-, 8-, 8.5-, 9-, 9.5-, or 10-fold enhanced or higher, or any value or range therein, as measured by relative Pi-Tou score of the substituted furin cleavage site as compared to the Pi-Tou score of an unsubstituted furin cleavage site in a corresponding wildtype flavivirus prM glycoprotein. In some embodiments, the furin cleavage site cleavability is enhanced about 1.5-fold to about 10-fold, about 2-fold to about 5-fold, about 1.5-fold to about 20-fold, or about 1.5-fold, about 2-fold, about 5-fold, about 10-fold, or about 20-fold or higher.

In some embodiments, the furin cleavage site has a Pi-Tou score of about 5 or higher, e.g., about 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, or higher, or any value or range therein. For example, in some embodiments, the furin cleavage site has a Pi-Tou score of about 5 to about 10, about 5 to about 20, about 6.5 to about 9, about 6.5 to about 15.5, about 10 to about 15.5, or about 5, about 6.5, about 9, about 10, about 15.5, or about 20.

In some embodiments, the recombinant flavivirus prM glycoprotein of the present invention may comprise the substitution in amino acid positions 83-92 of the amino acid sequence of any one of SEQ ID NO:1-103 or a sequence at least about 70% identical thereto (e.g., about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical thereto); wherein the numbering corresponds to the amino acid sequence of SEQ ID NO:104 [wt DENV2 prM]. In some embodiments, the substitution in amino acid positions 83-92 may comprise, consists essentially of, or consist of the amino acid sequence of any one of SEQ ID NO:1-103.

The flavivirus prM glycoprotein backbone of the present invention may be from any flavivirus known or later discovered. For example, in some embodiments, the prM glycoprotein backbone may comprise a backbone of a flavivirus such as, but not limited to, dengue virus (DENV, also referred to as DV), zika virus (ZIKV), West Nile virus (WNV), Japanese encephalitis virus (JEV), yellow fever virus (YFV), tick-borne encephalitis virus (TBEV), Kyasanur Forest disease virus (KFDV), Kunjin virus (KUN), Murray Valley encephalitis virus (MVEV), St. Louis encephalitis virus (SLEV), Powassan virus (POW), Usutu virus (USUV), and/or any derivative thereof. In some embodiments, the prM glycoprotein backbone may be a backbone of dengue virus serotype 1, 2, 3, 4, or any derivative thereof, e.g., a modified DV-1, -2, -3, -4, a chimeric DV, a fully synthetic DV, and the like.

A DENV1 backbone may comprise the amino acid sequence of any DENV1 genotype and/or strain and/or isolate currently known or as yet identified and/or isolated. Non-limiting examples of DENV1 genotypes, strains, and/or isolates include Genotypes I, II, III, IV, and V, strains Western Pacific 1974, and GenBank® Accession No. U88535.1.

A DENV3 backbone may comprise the amino acid sequence of any DENV3 genotype and/or strain and/or isolate currently known or as yet identified and/or isolated. Non-limiting examples of DENV3 genotypes, strains, and/or isolates include Genotype I, II, III, IV, strains such as Sri Lanka 1989, Indonesia 1982, Thailand 1995, Cuba 2002, and Puerto Rico 1977, and GenBank® Accession Nos. JQ411814.1 (“UNC3001”), DQ401690.1, AY676376, AY02031, and AY146761.

A DENV2 backbone may comprise the amino acid sequence of any DENV2 genotype and/or strain and/or isolate currently known or as yet identified and/or isolated. Non-limiting examples of DENV2 genotypes, strains, and/or isolates include Genotypes Cosmopolitan, Asian-American, Asian I, and Asian II, strains such as S-16803 (GenBank® Accession No. GU289914.1), India 2001 (GenBank® Accession No. DQ448237b), Puerto Rico 2003 (GenBank® Accession No. EU687235b), USA 2009 (GenBank® Accession No. HQ541798b), Indonesia 2016 (GenBank® Accession No. MH173162), Thailand 2016 (GenBank® Accession No. LC410185b), and GenBank® Accession No. NC_001474.2.

A DENV4 backbone may comprise the amino acid sequence of any DENV4 genotype and/or strain and/or isolate currently known or as yet identified and/or isolated. Non-limiting examples of DENV4 genotypes, strains, and/or isolates include Genotypes I, IIa, IIb, III, IV, and V and strains such as GenBank® Accession Nos. KF543272, JN832541, FJ882599, KJ160504.1, AY618940, AF231724, and JF262783.

In some embodiments, the recombinant flavivirus prM glycoprotein of the present invention may comprise, consist essentially of, or consist of an amino acid sequence at least 70% identical (e.g., at least about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% identical) to any one of the amino acid sequence of SEQ ID NO:109-116.

[DV2-V2 prM]
SEQ ID NO: 109.
FHLTTRNGEPHMIVSRQEKGKSLLFKTEDGVNMCTLMAMDLGELC
EDTITYNCPLLRQNEPEDIDCWCNSTSTWVTYGTCTTTGSKMRSK
RSVALVPHVGMGLETRTETWMSSEGAWKHAQRIETWILRHPGFTI
MAAILAYTIGTTHFQRALIFILLTAVAPSMT
DV2-C1 prM
SEQ ID NO: 110.
FHLTTRNGEPHMIVSRQEKGKSLLFKTEDGVNMCTLMAMDLGELC
EDTITYNCPLLRQNEPEDIDCWCNSTSTWVTYGTCTTTGRAQRYK
RSVALVPHVGMGLETRTETWMSSEGAWKHAQRIETWILRHPGFTI
MAAILAYTIGTTHFQRALIFILLTAVAPSMT
DV2-V1 prM
SEQ ID NO: 111.
FHLTTRNGEPHMIVSRQEKGKSLLFKTEDGVNMCTLMAMDLGELC
EDTITYNCPLLRQNEPEDIDCWCNSTSTWVTYGTCTTTGAGRRSK
RSVALVPHVGMGLETRTETWMSSEGAWKHAQRIETWILRHPGFTI
MAAILAYTIGTTHFQRALIFILLTAVAPSMT
DV1-prM-D89K
SEQ ID NO: 112.
FHLTTRGGEPHMIVSKQERGKSLLFKTSAGVNMCTLIAMDLGELC
EDTMTYKCPRITETEPDDVDCWCNATETWVTYGTCSQTGEHRRKK
RSVALAPHVGLGLETRTETWMSSEGAWKQIQKVETWALRHPGFTV
IALFLAHAIGTSITQKGIIFILLMLVTPSMA
DV2-prM-E89K
SEQ ID NO: 113.
FHLTTRNGEPHMIVSRQEKGKSLLFKTEDGVNMCTLMAMDLGELC
EDTITYNCPLLRQNEPEDIDCWCNSTSTWVTYGTCTTTGEHRRKK
RSVALVPHVGMGLETRTETWMSSEGAWKHAQRIETWILRHPGFTI
MAAILAYTIGTTHFQRALIFILLTAVAPSMT
DV3-prM-E89K
SEQ ID NO: 114.
FHLTSRDGEPRMIVGKNERGKSLLFKTASGINMCTLIAMDLGEMC
DDTVTYKCPHITEVEPEDIDCWCNLTSTWVTYGTCNQAGEHRRKK
RSVALAPHVGMGLDTRTQTWMSAEGAWRQVEKVETWALRHPGFTI
LALFLAHYIGTSLTQKVVIFILLMLVTPSMT
DV4-prM-E89K
SEQ ID NO: 115.
FHLSTRDGEPLMIVAKHERGRPLLFKTTEGINKCTLIAMDLGEMC
EDTVTYKCPLLVNTEPEDIDCWCNLTSTWVMYGTCTQSGERRRKK
RSVALTPHSGMGLETRAETWMSSEGAWKHAQRVESWILRNPGFAL
LAGFMAYMIGQTGIQRTVFFVLMMLVAPSYG
DV4-prM-E89N
SEQ ID NO: 116.
FHLSTRDGEPLMIVAKHERGRPLLFKTTEGINKCTLIAMDLGEMC
EDTVTYKCPLLVNTEPEDIDCWCNLTSTWVMYGTCTQSGERRRNK
RSVALTPHSGMGLETRAETWMSSEGAWKHAQRVESWILRNPGFAL
LAGFMAYMIGQTGIQRTVFFVLMMLVAPSYG

The amino acid residue positions of the substitutions that can be made to produce the desired recombinant prM glycoproteins of the present invention can be readily determined by one of ordinary skill in the art according to the teachings herein and according to protocols well known in the art. The amino acid residue numbering provided in the amino acid sequences set forth here is based on the reference sequence of DENV2 wild type prM glycoprotein, as provided herein (SEQ ID NO:104). However it would be readily understood by one of ordinary skill in the art that the equivalent amino acid positions in other flavivirus prM glycoproteins can be readily identified and employed in the production of the recombinant prM glycoproteins of this invention.

It would be understood that the modifications described above provide multiple examples of how the amino acid sequences described herein can be obtained and that, due to the degeneracy of the amino acid codons, numerous other modifications can be made to a nucleotide sequence encoding a prM glycoprotein or fragment thereof to obtain the desired amino acid sequence. The present invention provides additional non limiting examples of nucleic acids and/or polypeptides of this invention that can be used in the compositions and methods described herein in the SEQUENCES section provided herein.

The present invention further provides an isolated nucleic acid molecule encoding the recombinant flavivirus prM glycoprotein of this invention. In some embodiments, a nucleic acid molecule of this invention may be a cDNA molecule. In some embodiments, a nucleic acid molecule of this invention may be an mRNA molecule, e.g., an mRNA-based prophylactic and/or therapeutic immunogen (e.g., vaccine), e.g., an mRNA-lipid nanoparticle (mRNA-LNP) molecule.

Also provided is a vector, plasmid or other nucleic acid construct comprising the isolated nucleic acid molecule of this invention.

A vector can be any suitable means for delivering a polynucleotide to a cell. A vector of this invention can be an expression vector that contains all of the genetic components required for expression of the nucleic acid in cells into which the vector has been introduced, as are well known in the art. The expression vector can be a commercial expression vector or it can be constructed in the laboratory according to standard molecular biology protocols. The expression vector can comprise viral nucleic acid including, but not limited to, poxvirus, vaccinia virus, adenovirus, retrovirus, alphavirus and/or adeno-associated virus nucleic acid. The nucleic acid molecule or vector of this invention can also be in a liposome or a delivery vehicle, which can be taken up by a cell via receptor-mediated or other type of endocytosis. The nucleic acid molecule of this invention can be in a cell, which can be a cell expressing the nucleic acid whereby a recombinant prM glycoprotein of this invention is produced in the cell (e.g., a host cell). In addition, the vector of this invention can be in a cell, which can be a cell expressing the nucleic acid of the vector whereby a recombinant prM glycoprotein of this invention is produced in the cell. It is also contemplated that the nucleic acid molecules and/or vectors of this invention can be present in a host organism (e.g., a transgenic organism), which expresses the nucleic acids of this invention and produces a recombinant prM glycoprotein of this invention. In some embodiments, the vector is a plasmid, a viral vector, a bacterial vector, an expression cassette, a transformed cell, or a nanoparticle. For example, in some embodiments a recombinant prM glycoprotein of the present invention may be used in combination (e.g., in scaffold(s) and/or conjugated with) other molecules such as, but not limited to, nanoparticles, e.g., as delivery devices.

Types of nanoparticles of this invention for use as a vector and/or delivery device include, but are not limited to, polymer nanoparticles such as PLGA-based, PLA-based, polysaccharide-based (dextran, cyclodextrin, chitosan, heparin), dendrimer, hydrogel; lipid-based nanoparticles such as lipid nanoparticles, lipid hybrid nanoparticles, liposomes, micelles; inorganics-based nanoparticles such as superparamagnetic iron oxide nanoparticles, metal nanoparticles, platin nanoparticles, calcium phosphate nanoparticles, quantum dots; carbon-based nanoparticles such as fullerenes, carbon nanotubes; and protein-based complexes with nanoscales. Types of microparticles of this invention include but are not limited to particles with sizes at micrometer scale that are polymer microparticles including but not limited to, PLGA-based, PLA-based, polysaccharide-based (dextran, cyclodextrin, chitosan, heparin), dendrimer, hydrogel; lipid-based microparticles such as lipid microparticles, micelles; inorganics-based microparticles such as superparamagnetic iron oxide microparticles, platin microparticles and the like as are known in the art. These particles may be generated and/or have materials be absorbed, encapsulated, or chemically bound through known mechanisms in the art.

In some embodiments, a nanoparticle vector of the present invention may be an mRNA lipid nanoparticle (mRNA-LNP), a nucleic acid vaccine (NAV), or other nucleic acid lipid nanoparticle compositions, such as described in U.S. Pat. Nos. 9,868,692; 9,950,065; 10,041,091; 10,576,146; 10,702,600; WO2015/164674; US2019/0351048; US2020/297634; WO2020/097548; and Buschmann et al. 2021 Vaccines 9(65) doi.org/10.3390/vaccines9010065; Laczk6 et al. 2020 Immunity 53:724-732; and Pardi et al. 2018 Nat. Rev. Drug Discov. 17:261-279, the disclosures of each of which are incorporated herein by reference in their entireties.

It is known in the art that many attempts to produce flavivirus vaccines such as dengue virus vaccines result in the production of non-neutralizing antibodies, which may increase the likelihood of pathology upon subsequence exposure to natural infection or vaccine. Another approach to provide an engineered epitope is to deliver all or a portion of the flavivirus protein incorporated into another flavivirus particle or VLP. Portions of the flavivirus protein (e.g., prM glycoprotein, e.g., E glycoprotein, e.g., capsid protein) can be grafted into the relevant protein of the heterologous flavivirus backbone, e.g., to reduce the generation of non-neutralizing dengue virus antibodies to non-neutralizing epitopes present in the relevant flavivirus protein and/or other virus structural proteins.

Thus, a modified (e.g., recombinant and/or chimeric) flavivirus or flavivirus VLP can present the quaternary flavivirus epitope in proper conformation while reducing the generation of non-neutralizing antibodies to other portions of the flavivirus proteins that are not presented in the modified flavivirus or flavivirus VLP.

In some embodiments of the invention the individual and conformational epitopes of the flavivirus glycoproteins can be presented on a synthetic backbone or support structure so that the epitopes within the synthetic backbone or support structure mimic the conformation and arrangement of the epitopes within the structure of the source glycoprotein, virus particle or VLP.

In still further embodiments of the invention, the present invention provides peptide mimitopes (see, Meloen et al. (2000) J. Mol. Recognit. 13:352-359) that mimic the individual and conformational epitopes of the glycoproteins of the invention. Mimitopes may be identified using any technique known in the art, including but not limited to surface stimulation, random peptide libraries or phage display libraries, as well as an antibody or antibodies to the individual and conformational epitopes of the glycoproteins of the invention.

The invention further provides a nucleic acid (e.g., an isolated nucleic acid) encoding a recombinant flavivirus VLP or a recombinant flavivirus particle (e.g., a viral coat of the flavivirus particle) of the invention. In some embodiments, the flavivirus particle or virus like particle (VLP) comprises the prM glycoprotein of the present invention.

In some embodiments, a flavivirus particle or VLP of the present invention may comprise a mature form.

Flaviviruses such as dengue virus can adopt either a mature and/or an immature virion structure. As used herein, the term “mature form” refers to the infectious form of a viral particle structure, which in nature is formed from structural changes to the immature form during egress of the virus particle from infected cells. While not wishing to be bound to theory, flaviviruses typically assemble as an immature (e.g., non-infectious) form wherein the surface displayed structural proteins E and prM form trimeric spikes. During egress of the virus particle from the host (infected) cell, the prM protein is cleaved by furin enzyme and the pr component is released from the virus particle upon exposure to neutral pH of the extracellular space. This maturation process allows for structural reconfiguration of the surface proteins that assemble the mature form of a flavivirus virion as a herringbone arrangement of E glycoproteins which lie flat against the surface of the virion (Pierson and Diamond, 2012 Curr. Opin. Virol. 2:168-175).

For example, a mature form of a dengue virus particle comprises a structure of about 90 E glycoprotein homodimers lying flat in a herringbone structure, organized into a 50 nm icosahedral (T=pseudo 3) symmetry. In contrast to the mature form, a dengue virus immature form comprises a structure of a spikey sphere of about 60 nm, with 60 three-fold spikes. A third flavivirus virion form represents partially mature/immature particles which adopt both mature and immature structure in different regions of the particle. Populations of viral particles may comprise mature, immature, and/or partially mature/immature particles, in any combination, in any ratio. Maturation status and affects thereof on viral characteristics are further described in Kuhn et al. 2002 Cell 108:717-725; Zybert et al. 2008 J. Gen. Virol. 89:3047-3051; Plevka et al. 2014 J. Struct. Biol. 185:27-31; Kostyuchenko et al. 2013 J. Virol. 87:7700-7707, Pierson and Diamond, 2012 Curr. Opin. Virol. 2:168-175; and Galula et al. 2019 Hum. Vaccines Immunother. 15:2328-2336, incorporated herein by reference in their entireties.

Accordingly, in some embodiments of the present invention, the mature form of a virus particle comprises wherein the virus particle assembles in a herringbone structure formed from homodimers of E glycoproteins with icosahedral symmetry.

Also provided herein are populations of flavivirus particles of the present invention (e.g., in mature forms, e.g., comprising a recombinant flavivirus prM glycoprotein of the present invention).

In some embodiments, a greater percentage of the flavivirus particles in the population are in a mature form as compared to the percentage of flavivirus particles in a mature form in a population of corresponding wildtype flavivirus particles (e.g., wildtype DV2 particles). For example, in some embodiments, about 10%, 20%, 30%, 40%, 50%, 60%, 70%, 80%, 85%, 90%, 91%, 92%, 93%, 94%, 95%, 96%, 97%, 98%, 99%, or 100% or more or any value or range therein, of the flavivirus particles are in a mature form, as compared to the percentage of flavivirus particles in a mature form in a population of corresponding wildtype flavivirus particles.

The relative amount of mature forms versus immature forms of a population of flavivirus particles may be measured by any standard method in the art, as will be apparent to those skilled in the art in light of the instant disclosure. For example, in some embodiments, the amount of mature forms versus immature forms of a population of flavivirus particles may be measured by determining the ratio of prM glycoprotein versus E glycoprotein (prM/Env) in a population. In some embodiments, the ratio of prM glycoprotein versus E glycoprotein in the population may be less than 1.0 (e.g., 0.99, 0.98, 0.97, 0.96, 0.95, 0.9, 0.85, 0.8, 0.75, 0.7, 0.65, 0.6, 0.55, 0.5, 0.45, 0.4, 0.35, 0.3, 0.25, 0.2, 0.15, 0.1, 0.05, 0.04, 0.03, 0.02, 0.01, or less, or any value or range therein), normalized to the ratio of prM/Env in a population of corresponding wildtype flavivirus particles (e.g., wildtype DV2 particles; e.g., wherein the ratio of prM/Env in the population of the corresponding wildtype flavivirus particles is set to 1.0).

Also provided is a cell (e.g., an isolated cell) comprising a vector, a nucleic acid molecule, a dengue virus protein, a dengue virus peptide, a dengue virus protein domain, a flavivirus protein, a flavivirus peptide, flavivirus protein domain, a dengue virus particle, a dengue virus VLP, a flavivirus VLP and/or a flavivirus particle of this invention, singly or in any combination.

The invention also provides immunogenic compositions comprising the cells, vectors, nucleic acids molecules, dengue virus proteins, dengue virus VLPs, chimeric dengue virus particles, flavivirus VLPs and/or flavivirus particles of the invention, singly or in any combination. In some embodiments, the immunogenic composition is monovalent. In some embodiments, the immunogenic composition is multivalent (e.g., bivalent, trivalent or tetravalent) for flavivirus serotypes, e.g., dengue virus serotypes DENV1, DENV2, DENV3 and/or DENV4 in any combination. The recombinant flavivirus prM glycoproteins of this invention can be administered to a subject singly or in any combination, including any combination of priming and boosting according to such immunization protocols that are known in the art. In some embodiments, a prime/boost combination would be used that results in administration of antigens representative of all four dengue virus serotypes. Such a prime/boost regimen can include administration of any combination of antigens in any order to achieve this result. A nonlimiting example of a prime/boost protocol can include priming at day 0 and boosting at 3 months and 6 months, or boosting at 6 months and 1 year, respectively. This protocol could also be modified to include only one boost at either 3 months, 6 months or 1 year.

The invention also provides immunogenic compositions comprising the cells, vectors, nucleic acid molecules, VLPs, VRPs, dengue virus or flavivirus particles and/or populations of the invention. The composition can further comprise a pharmaceutically acceptable carrier.

By “pharmaceutically acceptable” it is meant a material that is not toxic or otherwise undesirable, i.e., the material may be administered to a subject without causing any undesirable biological effects. For injection, the carrier will typically be a liquid. For other methods of administration (e.g., such as, but not limited to, administration to the mucous membranes of a subject (e.g., via intranasal administration, buccal administration and/or inhalation)), the carrier may be either solid or liquid. For inhalation administration, the carrier will be respirable, and will preferably be in solid or liquid particulate form. The formulations may be conveniently prepared in unit dosage form and may be prepared by any of the methods well known in the art. In some embodiments, that pharmaceutically acceptable carrier can be a sterile solution or composition.

In some embodiments, the present invention provides a pharmaceutical composition comprising a dengue virus or flavivirus prM glycoprotein, nucleic acid molecule (e.g., an mRNA molecule), vector, VRP, VLP, flavivirus particle, population and/or composition of the present invention, a pharmaceutically acceptable carrier, and, optionally, other medicinal agents, therapeutic agents, pharmaceutical agents, stabilizing agents, buffers, carriers, adjuvants, diluents, etc., which can be included in the composition singly or in any combination and/or ratio.

Immunogenic compositions comprising dengue virus or flavivirus prM glycoprotein, nucleic acid molecule, vector, VRP, VLP, dengue virus or flavivirus particle, population and/or composition of the present invention may be formulated by any means known in the art. Such compositions, especially vaccines, are typically prepared as injectables, either as liquid solutions or suspensions. Solid forms suitable for solution in, or suspension in, liquid prior to injection may also be prepared. Lyophilized preparations are also suitable. In some embodiments, a pharmaceutical composition of the present invention may be a vaccine formulation, e.g., may comprise dengue virus or flavivirus protein, nucleic acid molecule, vector, VRP, VLP, flavivirus particle, population and/or composition of the present invention and adjuvant(s), optionally in a vaccine diluent. The active immunogenic ingredients are often mixed with excipients and/or carriers that are pharmaceutically acceptable and/or compatible with the active ingredient. Suitable excipients include but are not limited to sterile water, saline, dextrose, glycerol, ethanol, or the like and combinations thereof, as well as stabilizers, e.g., HSA or other suitable proteins and reducing sugars. In addition, if desired, the vaccines or immunogenic compositions may contain minor amounts of auxiliary substances such as wetting and/or emulsifying agents, pH buffering agents, and/or adjuvants that enhance the effectiveness of the vaccine or immunogenic composition.

In some embodiments, a pharmaceutical composition comprising dengue virus or flavivirus prM glycoprotein, nucleic acid molecule, vector, VRP, VLP, dengue virus or flavivirus particle, population and/or composition of the present invention may further comprise additional agents, such as, but not limited to, additional antigen as part of a cocktail in a vaccine, e.g., a multi-component vaccine wherein the vaccine may additionally include peptides, cells, virus, viral peptides, inactivated virus, etc. Thus, in some embodiments, a pharmaceutical composition comprising a dengue virus or flavivirus prM glycoprotein, nucleic acid molecule, vector, VRP, VLP, particle, population and/or composition of the present invention, a pharmaceutically acceptable carrier may further comprise additional viral antigen, e.g., a flavivirus antigen in the form of peptides, peptoids, whole flavivirus (e.g., live attenuated and/or inactivated virus), and/or flavivirus-comprising cells (e.g., cells modified to express flaviviral components, e.g., dengue or other flaviviral peptides).

In some embodiments, a pharmaceutical composition comprising a dengue virus or flavivirus prM glycoprotein, nucleic acid molecule, vector, VRP, VLP, particle, population and/or composition of the present invention, and a pharmaceutically acceptable carrier may further comprise an adjuvant. As used herein, “suitable adjuvant” describes an adjuvant capable of being combined with dengue virus or flavivirus prM glycoprotein, nucleic acid molecule, vector, VRP, VLP, particle, population and/or composition of this invention to further enhance an immune response without deleterious effect on the subject or the cell of the subject.

The adjuvants of the present invention can be in the form of an amino acid sequence, and/or in the form or a nucleic acid encoding an adjuvant. When in the form of a nucleic acid, the adjuvant can be a component of a nucleic acid encoding the polypeptide(s) or fragment(s) or epitope(s) and/or a separate component of the composition comprising the nucleic acid encoding the polypeptide(s) or fragment(s) or epitope(s) of the invention. According to the present invention, the adjuvant can also be an amino acid sequence that is a peptide, a protein fragment or a whole protein that functions as an adjuvant, and/or the adjuvant can be a nucleic acid encoding a peptide, protein fragment or whole protein that functions as an adjuvant. As used herein, “adjuvant” describes a substance, which can be any immunomodulating substance capable of being combined with a composition of the invention to enhance, improve, or otherwise modulate an immune response in a subject.

In further embodiments, the adjuvant can be, but is not limited to, an immunostimulatory cytokine (including, but not limited to, GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules), SYNTEX adjuvant formulation 1 (SAF-1) composed of 5 percent (wt/vol) squalene (DASF, Parsippany, N.J.), 2.5 percent Pluronic, L121 polymer (Aldrich Chemical, Milwaukee), and 0.2 percent polysorbate (Tween 80, Sigma) in phosphate-buffered saline. Suitable adjuvants also include an aluminum salt such as aluminum hydroxide gel (alum), aluminum phosphate, or algannmulin, but may also be a salt of calcium, iron or zinc, or may be an insoluble suspension of acylated tyrosine, or acylated sugars, cationically or anionically derivatized polysaccharides, or polyphosphazenes.

Other adjuvants are well known in the art and include without limitation MF 59, LT-K63, LT-R72 (Pal et al. Vaccine 24(6):766-75 (2005)), QS-21, Freund's adjuvant (complete and incomplete), aluminum hydroxide, N-acetyl-muramyl-L-threonyl-D-isoglutamine (thr-MDP), N-acetyl-normuramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE) and RIBI, which contains three components extracted from bacteria, monophosphoryl lipid A, trealose dimycolate and cell wall skeleton (MPL+TDM+CWS) in 2% squalene/Tween 80 emulsion.

Additional adjuvants can include, for example, a combination of monophosphoryl lipid A, preferably 3-de-O-acylated monophosphoryl. lipid A (3D-MPL) together with an aluminum salt. An enhanced adjuvant system involves the combination of a monophosphoryl lipid A and a saponin derivative, particularly the combination of QS21 and 3D-MPL as disclosed in PCT publication number WO 94/00153, or a less reactogenic composition where the QS21 is quenched with cholesterol as disclosed in PCT publication number WO 96/33739. A particularly potent adjuvant formulation involving QS21 3D-MPL & tocopherol in an oil in water emulsion is described in PCT publication number WO 95/17210. In addition, the nucleic acid compositions of the invention can include an adjuvant by comprising a nucleotide sequence encoding the antigen and a nucleotide sequence that provides an adjuvant function, such as CpG sequences. Such CpG sequences, or motifs, are well known in the art.

Adjuvants can be combined, either with the compositions of this invention or with other vaccine compositions that can be used in combination with the compositions of this invention.

### Methods of Using

The nucleic acids, proteins, peptides, viruses, vectors, particles, antibodies, VLPs, VRPs, populations, and/or compositions of this invention are intended for use as therapeutic agents and immunological reagents, for example, as antigens, immunogens, vaccines, and/or nucleic acid delivery vehicles. The compositions described herein can be formulated for use as reagents (e.g., to produce antibodies) and/or for administration in a pharmaceutical carrier in accordance with known techniques. See, e.g., Remington, The Science and Practice of Pharmacy (latest edition).

Accordingly, another aspect of the present invention provides a method of producing an immune response to a flavivirus in a subject, comprising administering to the subject an effective amount of a recombinant prM glycoprotein, flavivirus particle or VLP, nucleic acid molecule, vector, population, and/or composition of the invention, and any combination thereof.

Another aspect of the present invention provides a method of treating a flavivirus infection in a subject, comprising administering to the subject an effective amount of a recombinant prM glycoprotein, flavivirus particle or VLP, nucleic acid molecule, vector, population, and/or composition of the invention, and any combination thereof.

Another aspect of the present invention provides a method of preventing a disorder associated with flavivirus infection in a subject, comprising administering to the subject an effective amount of a recombinant prM glycoprotein, flavivirus particle or VLP, nucleic acid molecule, vector, population, and/or composition of the invention, and any combination thereof.

Another aspect of the present invention provides a method of protecting a subject from the effects of a flavivirus infection, comprising administering to the subject an effective amount of a recombinant prM glycoprotein, flavivirus particle or VLP, nucleic acid molecule, vector, population, and/or composition of the invention, and any combination thereof.

By “protecting a subject from the effects of a flavivirus infection” it is meant that the subject does not develop a disease or disorder caused by a flavivirus infection, or if the subject does develop a disease or disorder caused by a flavivirus infection, the disease or disorder is of less severity and/or symptoms are reduced and/or less severe in the subject in comparison to what the subject would experience upon infection by a flavivirus in the absence of the administration of the flavivirus protein, a flavivirus protein domain, a flavivirus peptide, a flavivirus particle, a flavivirus VLP, a nucleic acid molecule, a vector, a cell, and/or immunogenic composition of this invention.

Another aspect of the present invention provides a method of identifying the presence of a maturation-dependent neutralizing antibody to a flavivirus in a biological sample from a subject, comprising: a) contacting a biological sample from the subject with the flavivirus particle or VLP of the present invention (e.g., comprising a recombinant prM glycoprotein of the present invention), a composition comprising the same and/or an isolated nucleic acid encoding the same, in any combination, under conditions whereby neutralization of the flavivirus particles can be detected; and b) detecting neutralization in step (a), thereby identifying the presence of a maturation-dependent neutralizing antibody to the flavivirus in the biological sample from the subject.

Another aspect of the present invention provides a method of identifying the presence of a maturation-dependent neutralizing antibody to a flavivirus in a biological sample from a subject, comprising: a) contacting a biological sample from a subject that has been administered an E glycoprotein of a flavivirus (e.g., an isolated E glycoprotein and/or a flavivirus comprising an E glycoprotein) with flavivirus particles comprising the recombinant prM glycoprotein of the invention under conditions whereby neutralization of the flavivirus particles can be detected; and b) detecting neutralization in step (a), thereby identifying the presence of a maturation-dependent neutralizing antibody to the flavivirus in the biological sample from the subject.

Also provided herein is a method of identifying an immunogenic composition that induces an antibody to a flavivirus (e.g., dengue virus serotype 1, 2, 3, and/or 4) in a subject, the method comprising: a) contacting a biological sample from a subject that has been administered an immunogenic composition comprising a prM glycoprotein of the present invention and/or a mature flavivirus particle of the present invention, under conditions whereby an antigen/antibody complex can form; and b) detecting formation of an antigen/antibody complex, thereby identifying an immunogenic composition that induces an antibody to the flavivirus in the subject.

Regarding dengue viruses, there are four serotypes of dengue virus (DENV-1, DENV-2, DENV-3 and DENV-4). Within each serotype there are a number of different strains or genotypes. The dengue virus antigens and epitopes of the invention can be derived from any dengue virus, including all serotypes, strains and genotypes, now known or later identified.

In some embodiments of the invention, the dengue virus may be UNC1017 strain (DENV1), West Pacific 74 strain (DENV1), S16803 strain (DENV2), UNC2005 strain (DENV2), UNC3001 strain (DENV3), UNC3043 (DENV3 strain 059.AP-2 from Philippines, 1984), UNC3009 strain (DENV3, D2863, Sri Lanka 1989), UNC3066 (DENV3, strain 1342 from Puerto Rico 1977), CH53489 strain (DENV3), Indonesia 1982 (DENV3), Cuba 2002 (DENV3), UNC4019 strain (DENV4), or TVP-360 (DENV4).

The present invention provides additional non limiting examples of recombinant flavivirus prM glycoproteins of this invention that can be used in the compositions and methods described herein in the SEQUENCES section provided herein.

Another aspect of the present invention provides a method of detecting an antibody to a flavivirus in a sample, comprising: a) contacting the sample with the flavivirus particle or VLP of the present invention (e.g., comprising a recombinant prM glycoprotein of the present invention), a composition comprising the same and/or an isolated nucleic acid encoding the same, in any combination, under conditions whereby an antigen/antibody complex can form; and b) detecting formation of an antigen/antibody complex, thereby detecting an antibody to the flavivirus in the sample.

Another aspect of the present invention provides a method of detecting an antibody to a flavivirus in a sample, comprising: a) contacting the sample with the recombinant prM glycoprotein of the invention under conditions whereby an antigen/antibody complex can form; and b) detecting formation of an antigen/antibody complex, thereby detecting an antibody to the flavivirus in the sample.

In some embodiments of the methods disclosed herein, the sample is a biological sample from a subject.

The present invention can be practiced for prophylactic, therapeutic and/or diagnostic purposes. In addition, the invention can be practiced to produce antibodies for any purpose, such as diagnostic or research purposes, or for passive immunization by transfer to another subject.

For example, the recombinant prM proteins and/or mature flavivirus particles of this invention can be used to immunize a subject against infection by a new flavivirus, as well as treat a subject infected with a newly emerging flavivirus.

The recombinant flavivirus prM glycoprotein of the present invention may be administered in any frequency, amount, and/or route as needed to elicit an effective prophylactic and/or therapeutic effect in a subject (e.g., in a subject in need thereof) as described herein. In certain embodiments, the recombinant flavivirus prM glycoprotein, nucleic acid molecule, vector, VRP, VLP, flavivirus particle, population and/or composition is administered/delivered to the subject, e.g., systemically (e.g., intravenously). In particular embodiments, more than one administration (e.g., two, three, four or more administrations) may be employed to achieve the desired level of protein expression over a period of various intervals, e.g., daily, weekly, monthly, yearly, etc. The most suitable route in any given case will depend on the nature and severity of the condition being treated and on the nature of the particular delivery method that is being used. In embodiments wherein a vector is used, the vector will typically be administered in a liquid formulation by direct injection (e.g., stereotactic injection) to the desired region or tissues. In some embodiments, the vector can be delivered via a reservoir and/or pump. In other embodiments, the vector may be provided by topical application to the desired region or by intra-nasal administration of an aerosol formulation. Administration to the eye or into the ear, may be by topical application of liquid droplets. As a further alternative, the vector may be administered as a solid, slow-release formulation. For example, controlled release of parvovirus and AAV vectors is described in international patent publication WO 01/91803, which is incorporated by reference herein for these teachings.

Administration may be by any suitable means, such as intraperitoneally, intramuscularly, intranasally, intravenously, intradermally (e.g., by a gene gun), intrarectally and/or subcutaneously. The compositions herein may be administered via a skin scarification method, and/or transdermally via a patch or liquid. The compositions can be delivered subdermally in the form of a biodegradable material that releases the compositions over a period of time. As further non-limiting examples, the route of administration can be by inhalation (e.g., oral and/or nasal inhalation), oral, buccal (e.g., sublingual), rectal, vaginal, topical (including administration to the airways), intraocular, by parenteral (e.g., intramuscular [e.g., administration to skeletal muscle], intravenous, intra-arterial, intraperitoneal and the like), subcutaneous (including administration into the footpad), intrapleural, intracerebral, intrathecal, intraventricular, intra-aural, intra-ocular (e.g., intra-vitreous, sub-retinal, anterior chamber) and peri-ocular (e.g., sub-Tenon's region) routes or any combination thereof.

In some embodiments, recombinant flavivirus prM glycoprotein and/or mature flavivirus particle can be administered to a subject as a nucleic acid molecule, which can be a naked nucleic acid molecule or a nucleic acid molecule present in a vector (e.g., a delivery vector, which in some embodiments can be a viral vector, such as a VRP). The nucleic acids and vectors of this invention can be administered orally, intranasally, parenterally (e.g., intravenously), by intramuscular injection, by intraperitoneal injection, transdermally, extracorporeally, topically or the like. In the methods described herein which include the administration and uptake of exogenous DNA into the cells of a subject (i.e., gene transduction or transfection), the nucleic acids of the present invention can be in the form of naked DNA or the nucleic acids can be in a vector for delivering the nucleic acids to the cells for expression of the polypeptides and/or fragments of this invention. The vector can be a commercially available preparation or can be constructed in the laboratory according to methods well known in the art.

Delivery of the nucleic acid or vector to cells can be via a variety of mechanisms, including but not limited to recombinant vectors including bacterial, viral, and fungal vectors, liposomal delivery agents, nanoparticles, and gene gun related mechanisms.

In some embodiments, the nucleic acid molecules encoding recombinant flavivirus prM glycoprotein of this invention can be part of a recombinant nucleic acid construct comprising any combination of restriction sites and/or functional elements as are well known in the art that facilitate molecular cloning and other recombinant nucleic acid manipulations. Thus, the present invention further provides a recombinant nucleic acid construct comprising a nucleic acid molecule encoding recombinant flavivirus prM glycoprotein of this invention. The nucleic acid molecule encoding recombinant flavivirus prM glycoprotein of this invention can be any nucleic acid molecule that functionally encodes the recombinant flavivirus prM glycoprotein of this invention. To functionally encode recombinant flavivirus prM glycoprotein (i.e., allow the nucleic acids to be expressed), the nucleic acid of this invention can include, for example, expression control sequences, such as an origin of replication, a promoter, an enhancer and necessary information processing sites, such as ribosome binding sites, RNA splice sites, polyadenylation sites and transcriptional terminator sequences.

Non-limiting examples of expression control sequences that can be present in a nucleic acid molecule of this invention include promoters derived from metallothionine genes, actin genes, immunoglobulin genes, CMV, SV40, adenovirus, bovine papilloma virus, etc. A nucleic acid molecule encoding a selected recombinant prM glycoprotein can readily be determined based upon the genetic code for the amino acid sequence of the selected polypeptide and/or fragment of interest included in the recombinant prM glycoprotein, and many nucleic acids will encode any selected polypeptide and/or fragment. Modifications in the nucleic acid sequence encoding the polypeptide and/or fragment are also contemplated. Modifications that can be useful are modifications to the sequences controlling expression of the polypeptide and/or fragment to make production of the polypeptide and/or fragment inducible or repressible as controlled by the appropriate inducer or repressor. Such methods are standard in the art. The nucleic acid molecule and/or vector of this invention can be generated by means standard in the art, such as by recombinant nucleic acid techniques and/or by synthetic nucleic acid synthesis or in vitro enzymatic synthesis.

The nucleic acids and/or vectors of this invention can be transferred into a host cell (e.g., a prokaryotic or eukaryotic cell) by well-known methods, which vary depending on the type of cell host. For example, calcium chloride transfection is commonly used for prokaryotic cells, whereas calcium phosphate treatment, transduction, cationic lipid treatment and/or electroporation can be used for other cell hosts.

As another example, delivery can be via a liposome, using commercially available liposome preparations such as LIPOFECTIN, LIPOFECTAMINE (GIBCO-BRL, Inc., Gaithersburg, MD), SUPERFECT (Qiagen, Inc. Hilden, Germany) and TRANSFECTAM (Promega, Madison, WI), as well as other liposomes developed according to procedures standard in the art. In addition, the nucleic acid or vector of this invention can be delivered in vivo by electroporation, the technology for which is available from Genetronics, Inc. (San Diego, CA) as well as by means of a SONOPORATION machine (ImaRx Pharmaceutical Corp., Tucson, AZ).

As another example, vector delivery can be via a viral system, such as a retroviral vector system, which can package a recombinant retroviral genome. The recombinant retrovirus can then be used to infect and thereby deliver to the infected cells nucleic acid encoding the polypeptide and/or fragment of this invention. The exact method of introducing the exogenous nucleic acid into mammalian cells is, of course, not limited to the use of retroviral vectors. Other techniques are widely available for this procedure including the use of adenoviral vectors, alphaviral vectors (e.g., VRPs), adeno-associated viral (AAV) vectors, lentiviral vectors, pseudotyped retroviral vectors and vaccinia viral vectors, as well as any other viral vectors now known or developed in the future. Physical transduction techniques can also be used, such as liposome delivery and receptor-mediated and other endocytosis mechanisms. This invention can be used in conjunction with any of these or other commonly used gene transfer methods.

### Methods of Making

Another aspect of the present invention provides a method of producing a mature flavivirus particle, comprising: constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); thereby producing a mature flavivirus particle.

Another aspect of the present invention provides a method of increasing production of a mature form of a flavivirus particle, comprising: constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); thereby increasing production of the mature form of the flavivirus particle.

Another aspect of the present invention provides a method of enhancing antigenicity of a flavivirus particle, comprising: constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); thereby enhancing the antigenicity of the flavivirus particle.

Another aspect of the present invention provides a method of enhancing infectivity of a flavivirus particle, comprising: constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); thereby enhancing the infectivity of the flavivirus particle.

In some embodiments of the methods disclosed herein, the flavivirus particle may comprise a population of flavivirus particles.

Accordingly, another aspect of the present invention provides a method of enhancing antigenicity of a population of flavivirus particles, comprising: (a) constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); and (b) producing a population of the flavivirus particle of (a) (e.g., culturing the flavivirus particle, e.g., culturing in insect cell cultures and/or mammalian cell cultures, e.g., Vero cells, PDK-53 cells, C6/36 cells, VF1 cells); thereby enhancing the antigenicity of the population of flavivirus particles.

Another aspect of the present invention provides a method of enhancing infectivity of a population of flavivirus particles, (a) constructing a flavivirus particle comprising a substituted prM glycoprotein of the present invention (e.g., a recombinant prM glycoprotein of the present invention), wherein the presence of the substituted prM glycoprotein induces the flavivirus particle to assemble in a mature form (e.g., assemble in a herringbone structure formed from homodimers of E glycoproteins with icosahedral symmetry); and (b) producing a population of the flavivirus particle of (b) (e.g., culturing the flavivirus particle, e.g., culturing in insect cell cultures and/or mammalian cell cultures, e.g., Vero cells, PDK-53 cells, C6/36 cells, VF1 cells); thereby enhancing the infectivity of the population of flavivirus particles.

The recombinant prM glycoproteins of the present invention may be substituted by any standard known or later developed method for mutagenesis, as would be understood by the skilled artisan upon review of the disclosures herein. Non-limiting examples of mutagenesis methods include, but are limited to, site-directed mutagenesis, saturation mutagenesis, directed evolution, deep mutagenesis, unnatural amino acid incorporation, post-translational modification, or any combination thereof.

### Kits and Pharmaceutical Formulations

The present invention further provides a kit comprising one or more compositions of this invention. It would be well understood by one of ordinary skill in the art that the kit of this invention can comprise one or more containers and/or receptacles to hold the reagents (e.g., antibodies, antigens, nucleic acids) of the kit, along with appropriate buffers and/or diluents and/or other solutions and directions for using the kit, as would be well known in the art. Such kits can further comprise adjuvants and/or other immunostimulatory or immunomodulating agents, as are well known in the art.

The compositions and kits of the present invention can also include other medicinal agents, pharmaceutical agents, carriers, diluents, immunostimulatory cytokines, etc. Actual methods of preparing such dosage forms are known, or will be apparent, to those skilled in this art.

Immunomodulatory compounds, such as immunomodulatory chemokines and cytokines (preferably, CTL inductive cytokines) can be administered concurrently to a subject.

Cytokines may be administered by any method known in the art. Exogenous cytokines may be administered to the subject, or alternatively, a nucleic acid encoding a cytokine may be delivered to the subject using a suitable vector, and the cytokine produced in vivo. In particular embodiments, a viral adjuvant expresses the cytokine.

In embodiments of the invention, multiple dosages (e.g., two, three or more) of a composition of the invention can be administered without detectable pathogenicity (e.g., Dengue Shock Syndrome/Dengue Hemorrhagic Fever).

If ex vivo methods are employed, cells or tissues can be removed and maintained outside the body according to standard protocols well known in the art. The nucleic acids and vectors of this invention can be introduced into the cells via any gene transfer mechanism, such as, for example, virus-mediated gene delivery, calcium phosphate mediated gene delivery, electroporation, microinjection or proteoliposomes. The transduced cells can then be infused (e.g., in a pharmaceutically acceptable carrier) or transplanted back into the subject per standard methods for the cell or tissue type. Standard methods are known for transplantation or infusion of various cells into a subject.

In embodiments of the invention, the multivalent vaccines of the invention do not result in immune interference, e.g., a balanced immune response is induced against all antigens presented. In embodiments of the invention, the balanced response results in protective immunity against DENV-1, DENV-2, DENV-3 and DENV-4.

In embodiments of the invention, the multivalent vaccine can be administered to a subject that has anti-dengue maternal antibodies present.

An adjuvant for use with the present invention, such as any adjuvant disclosed herein, for example, an immunostimulatory cytokine, can be administered before, concurrent with, and/or within a few hours, several hours, and/or 1, 2, 3, 4, 5, 6, 7, 8, 9, and/or 10 days before and/or after the administration of a composition of the invention to a subject.

Furthermore, any combination of adjuvants, such as immunostimulatory cytokines, can be co-administered to the subject before, after and/or concurrent with the administration of an immunogenic composition of the invention. For example, combinations of immunostimulatory cytokines, can consist of two or more immunostimulatory cytokines, such as GM/CSF, interleukin-2, interleukin-12, interferon-gamma, interleukin-4, tumor necrosis factor-alpha, interleukin-1, hematopoietic factor flt3L, CD40L, B7.1 co-stimulatory molecules and B7.2 co-stimulatory molecules. The effectiveness of an adjuvant or combination of adjuvants can be determined by measuring the immune response produced in response to administration of a composition of this invention to a subject with and without the adjuvant or combination of adjuvants, using standard procedures, as described herein and as known in the art.

In embodiments of the invention, the adjuvant comprises an alphavirus adjuvant as described, for example in U.S. Pat. No. 7,862,829.

Boosting dosages can further be administered over a time course of days, weeks, months or years. In chronic infection, initial high doses followed by boosting doses may be advantageous.

The pharmaceutical formulations of the invention can optionally comprise other medicinal agents, pharmaceutical agents, stabilizing agents, buffers, carriers, diluents, salts, tonicity adjusting agents, wetting agents, and the like, for example, sodium acetate, sodium lactate, sodium chloride, potassium chloride, calcium chloride, sorbitan monolaurate, triethanolamine oleate, etc.

For injection, the carrier will typically be a liquid. For other methods of administration, the carrier may be either solid or liquid. For inhalation administration, the carrier will be respirable, and is typically in a solid or liquid particulate form.

The compositions of the invention can be formulated for administration in a pharmaceutical carrier in accordance with known techniques. See, e.g., Remington, The Science And Practice of Pharmacy (9^{th} Ed. 1995). In the manufacture of a pharmaceutical composition according to the invention, the VLPs are typically admixed with, inter alia, an acceptable carrier. The carrier can be a solid or a liquid, or both, and is optionally formulated with the compound as a unit-dose formulation, for example, a tablet. A variety of pharmaceutically acceptable aqueous carriers can be used, e.g., water, buffered water, 0.9% saline, 0.3% glycine, hyaluronic acid, pyrogen-free water, pyrogen-free phosphate-buffered saline solution, bacteriostatic water, or Cremophor EL[R] (BASF, Parsippany, N.J.), and the like. These compositions can be sterilized by conventional techniques. The formulations of the invention can be prepared by any of the well-known techniques of pharmacy.

The pharmaceutical formulations can be packaged for use as is, or lyophilized, the lyophilized preparation generally being combined with a sterile aqueous solution prior to administration. The compositions can further be packaged in unit/dose or multi-dose containers, for example, in sealed ampoules and vials.

The pharmaceutical formulations can be formulated for administration by any method known in the art according to conventional techniques of pharmacy. For example, the compositions can be formulated to be administered intranasally, by inhalation (e.g., oral inhalation), orally, buccally (e.g., sublingually), rectally, vaginally, topically, intrathecally, intraocularly, transdermally, by parenteral administration (e.g., intramuscular [e.g., skeletal muscle], intravenous, subcutaneous, intradermal, intrapleural, intracerebral and intra-arterial, intrathecal), or topically (e.g., to both skin and mucosal surfaces, including airway surfaces).

For intranasal or inhalation administration, the pharmaceutical formulation can be formulated as an aerosol (this term including both liquid and dry powder aerosols). For example, the pharmaceutical formulation can be provided in a finely divided form along with a surfactant and propellant. Typical percentages of the composition are 0.01-20% by weight, preferably 1-10%. The surfactant is generally nontoxic and soluble in the propellant. Representative of such agents are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides may be employed. The surfactant may constitute 0.1-20% by weight of the composition, preferably 0.25-5%. The balance of the composition is ordinarily propellant. A carrier can also be included, if desired, as with lecithin for intranasal delivery. Aerosols of liquid particles can be produced by any suitable means, such as with a pressure-driven aerosol nebulizer or an ultrasonic nebulizer, as is known to those of skill in the art. See, e.g., U.S. Pat. No. 4,501,729. Aerosols of solid particles can likewise be produced with any solid particulate medicament aerosol generator, by techniques known in the pharmaceutical art. Intranasal administration can also be by droplet administration to a nasal surface.

Injectable formulations can be prepared in conventional forms, either as liquid solutions or suspensions, solid forms suitable for solution or suspension in liquid prior to injection, or as emulsions. Alternatively, one can administer the pharmaceutical formulations in a local rather than systemic manner, for example, in a depot or sustained-release formulation.

Extemporaneous injection solutions and suspensions can be prepared from sterile powders, granules and tablets of the kind previously described. For example, an injectable, stable, sterile formulation of the invention in a unit dosage form in a sealed container can be provided. The formulation can be provided in the form of a lyophilizate, which can be reconstituted with a suitable pharmaceutically acceptable carrier to form a liquid composition suitable for injection into a subject. The unit dosage form can be from about 1 μg to about 10 grams of the formulation. When the formulation is substantially water-insoluble, a sufficient amount of emulsifying agent, which is pharmaceutically acceptable, can be included in sufficient quantity to emulsify the formulation in an aqueous carrier. One such useful emulsifying agent is phosphatidyl choline.

Pharmaceutical formulations suitable for oral administration can be presented in discrete units, such as capsules, cachets, lozenges, or tables, as a powder or granules; as a solution or a suspension in an aqueous or non-aqueous liquid; or as an oil-in-water or water-in-oil emulsion. Oral delivery can be performed by complexing a compound(s) of the present invention to a carrier capable of withstanding degradation by digestive enzymes in the gut of an animal. Examples of such carriers include plastic capsules or tablets, as known in the art. Such formulations are prepared by any suitable method of pharmacy, which includes the step of bringing into association the protein(s) and a suitable carrier (which may contain one or more accessory ingredients as noted above). In general, the pharmaceutical formulations are prepared by uniformly and intimately admixing the compound(s) with a liquid or finely divided solid carrier, or both, and then, if necessary, shaping the resulting mixture. For example, a tablet can be prepared by compressing or molding a powder or granules, optionally with one or more accessory ingredients. Compressed tablets are prepared by compressing, in a suitable machine, the formulation in a free-flowing form, such as a powder or granules optionally mixed with a binder, lubricant, inert diluent, and/or surface active/dispersing agent(s). Molded tablets are made by molding, in a suitable machine, the powdered protein moistened with an inert liquid binder.

Pharmaceutical formulations suitable for buccal (sub-lingual) administration include lozenges comprising the compound(s) in a flavored base, usually sucrose and acacia or tragacanth; and pastilles in an inert base such as gelatin and glycerin or sucrose and acacia.

Pharmaceutical formulations suitable for parenteral administration can comprise sterile aqueous and non-aqueous injection solutions, which preparations are preferably isotonic with the blood of the intended recipient. These preparations can contain anti-oxidants, buffers, bacteriostats and solutes, which render the composition isotonic with the blood of the intended recipient. Aqueous and non-aqueous sterile suspensions, solutions and emulsions can include suspending agents and thickening agents. Examples of nonaqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Pharmaceutical formulations suitable for rectal administration are optionally presented as unit dose suppositories. These can be prepared by admixing the active agent with one or more conventional solid carriers, such as for example, cocoa butter and then shaping the resulting mixture.

Pharmaceutical formulations suitable for topical application to the skin preferably take the form of an ointment, cream, lotion, paste, gel, spray, aerosol, or oil. Carriers that can be used include, but are not limited to, petroleum jelly, lanoline, polyethylene glycols, alcohols, transdermal enhancers, and combinations of two or more thereof. In some embodiments, for example, topical delivery can be performed by mixing a pharmaceutical formulation of the present invention with a lipophilic reagent (e.g., DMSO) that is capable of passing into the skin.

Pharmaceutical formulations suitable for transdermal administration can be in the form of discrete patches adapted to remain in intimate contact with the epidermis of the subject for a prolonged period of time. Formulations suitable for transdermal administration can also be delivered by iontophoresis (see, for example, Pharmaceutical Research 3:318 (1986)) and typically take the form of a buffered aqueous solution of the compound(s). Suitable formulations can comprise citrate or bis\tris buffer (pH 6) or ethanol/water and can contain from 0.1 to 0.2M active ingredient.

In embodiments of the invention, the dosage of a virus particle of this invention can be in a range of about 104 to about 107 plaque forming units (PFUs). In embodiments of this invention, the dosage of a VLP of this invention can be in a range of about 500 micrograms to about 5 milligrams. In embodiments of this invention, the dosage of a protein of this invention can be in a range of about 10⁰ to about 10⁴ micrograms +/−adjuvant.

Further, the composition can be formulated as a liposomal formulation. The lipid layer employed can be of any conventional composition and can either contain cholesterol or can be cholesterol-free. The liposomes that are produced can be reduced in size, for example, through the use of standard sonication and homogenization techniques.

The liposomal formulations can be lyophilized to produce a lyophilizate which can be reconstituted with a pharmaceutically acceptable carrier, such as water, to regenerate a liposomal suspension.

The immunogenic formulations of the invention can optionally be sterile, and can further be provided in a closed pathogen-impermeable container.

The invention will now be described with reference to the following examples. It should be appreciated that these examples are not intended to limit the scope of the claims to the invention but are rather intended to be exemplary of certain embodiments. Any variations in the exemplified methods that occur to the skilled artisan are intended to fall within the scope of the invention.

### Examples

### Example 1: DENV Mature Viruses

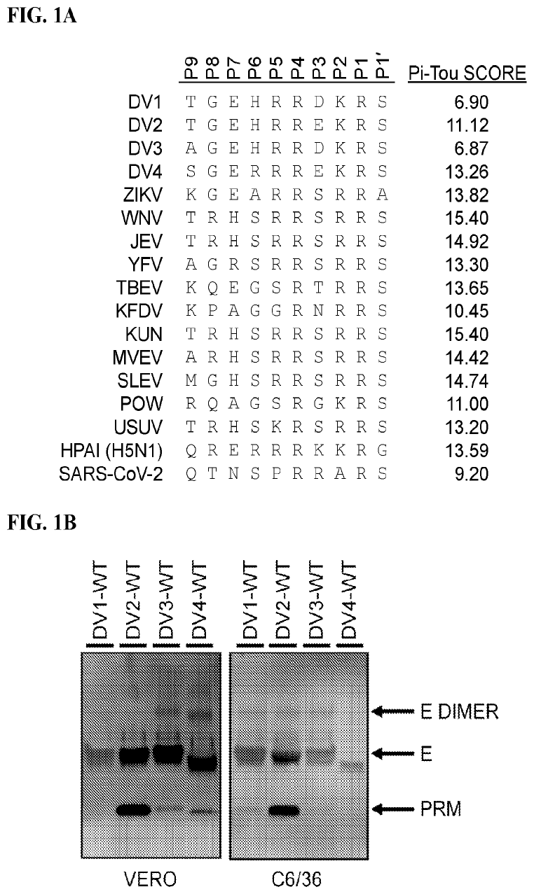

DENV Maturity is Serotype and Host Dependent. DENV maturation regulates virion infectivity and antigenicity and directly impacts antibody neutralization and potential vaccine efficacy. Because furin cleavage of the prM protein initiates the DENV maturation process, we hypothesized that furin cleavage efficiency is a major driver of DENV maturation. The DENV1-4 prM cleavage site was compared with other vector-borne Flaviviruses. Sequence analyses suggested that all the DENV serotypes encoded a sub-optimal furin cleavage site (P4) R-X-K/R-R (P1) with negative modulators as indicated by an acidic residue at the P3 position (FIG. 1A). To analyze the functionality of the prM furin cleavage site in a more quantitative manner, the computational program PiTou (www.nuolan.net/reference.html) was used, which calculates the logarithmic-odd probabilities of different viral furin cleavage sites by mammalian furin (Tian et al. 2012 Sci. Rep. 2:261, incorporated herein by reference). These analyses provided a reference that the DENV serotypes encode a less optimal furin sites (scores from 6.90-13.26) compared with other Flaviviruses (scores from 13.30-15.40) (FIG. 1A). We focused our studies on four prototypical wildtype (WT) DENV viruses including WestPac (DV1-WT), S16803 (DV2-WT), 3001 (DV3-WT) and Sri Lanka 92 (DV4-WT) isolates (Table 1). Using western blotting as a readout, we determined the relative maturity of each serotype by calculating the ratio of prM to E. Relative maturity was clearly different between serotypes. In particular, serotypes encoding a glutamic acid (E) at the P3 position (prM residue 89) were associated with more immature virion production in Vero cells, with DV2-WT virions containing the highest level of uncleaved prM, followed by DV4-WT, DV3-WT, and DV1-WT which had nearly undetectable levels of prM, and hence is more mature (FIG. 1B). DENV maturation also depends on the producer cells; C6/36 grown DENVs show a different maturation profile, wherein DV4-WT is the most mature (FIG. 1B). PiTou predictions do not perfectly translate to the actual maturation status of DENV, indicating that prM cleavage is dependent on both local primary sequence and other distal and structural influences. As such, PiTou scores serve as a reference.

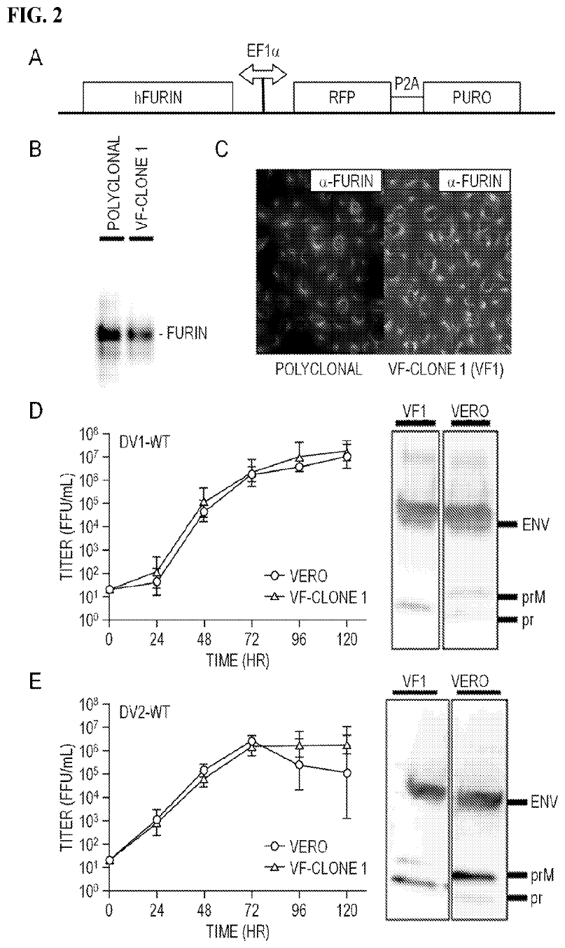

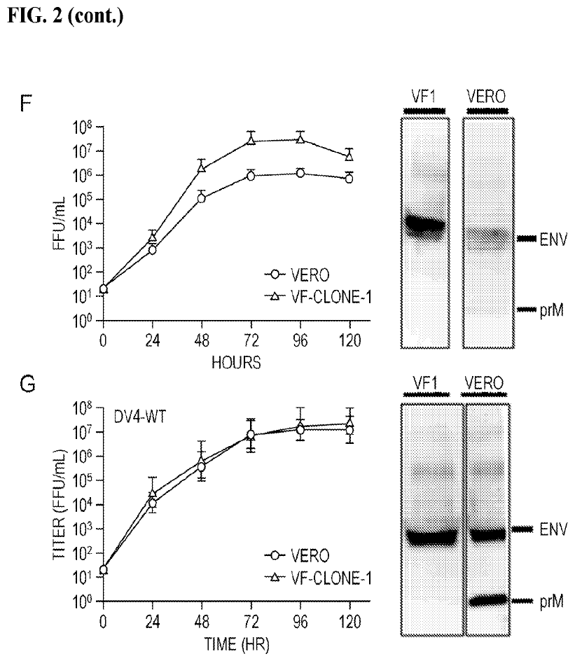

Clonal Vero-furin (VF) Cells Generate High Yield, Phenotypic Mature DENV. Fully mature DENV have been generated in Vero cells that overexpress furin (Mukherjee et al. 2016 Virology 497:33-40). Using the sleeping beauty transposon system, we generated a polyclonal cell line and subsequently a clonal line (VF1) with high levels of furin expression (FIG. 2, panel A). Immunofluorescent staining and western blot analysis revealed variable levels of furin expression in the trans-Golgi network of the polyclonal line but a uniform expression of the VF1 (FIG. 2, panels B and C). The growth kinetics of all four DENV serotypes were tested on VF1 line and compared to unmodified Vero cells (FIG. 2, panels D to G). DV1-WT, DV2-WT and DV4-WT showed similar growth kinetics in all cell lines tested, while VF1 supported better DV3-WT growth. VF1 supports the production of fully mature DENV virions across all four serotypes. Since the utility and reliability of previous furin-expressing lines has been limited by mycoplasma contamination, we confirmed that VF1 tested negative for mycoplasma contamination commercially.

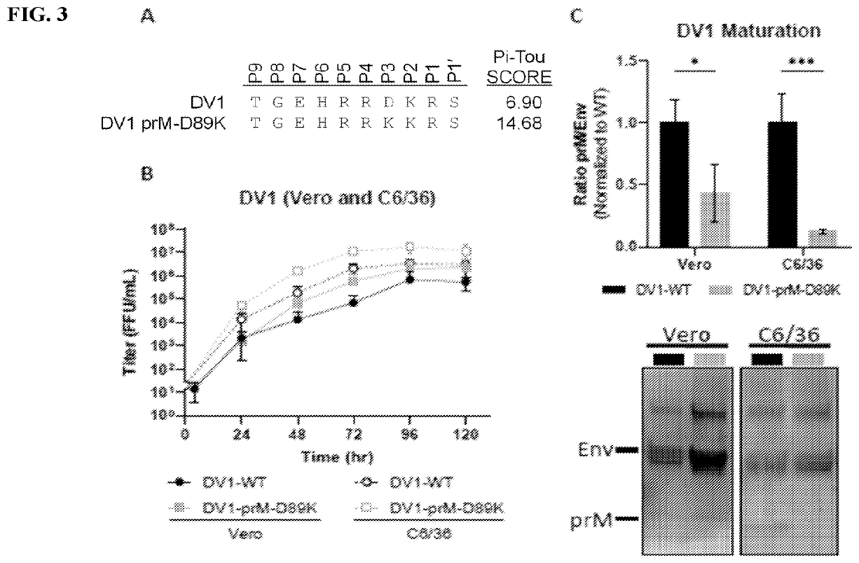

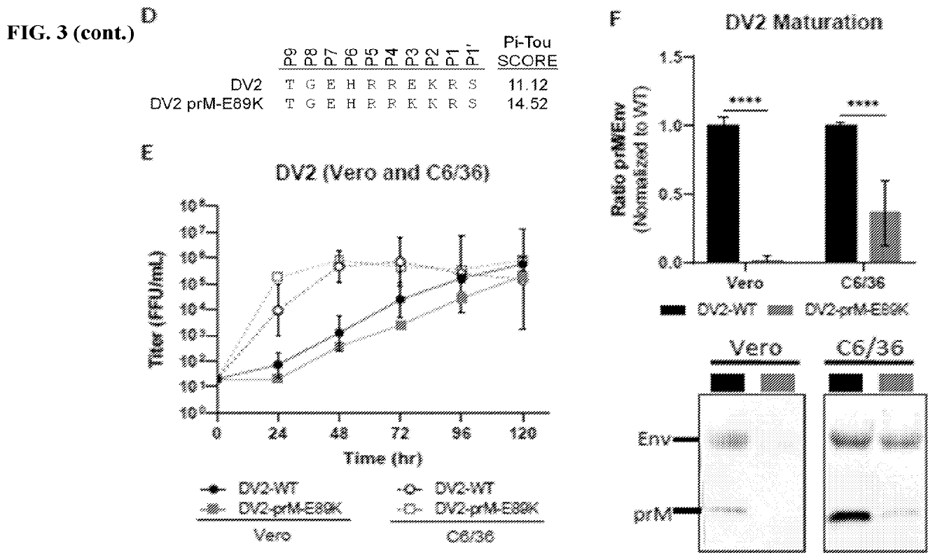

Genetic Enhancement of DENV prM Cleavage Sites Generates Mature DENV1 and DENV2. An alternative to ectopic overexpression of furin is genetic optimization of the DENV2 prM furin cleavage. To ensure this type of modification is transferable to all serotypes, we systemically introduced a furin enhancing mutation by substituting a favorable basic lysine (K) residue for an acidic aspartic acid (D) or glutamic acid (E) in the P3 position in all DENV serotypes. While not wishing to be bound to theory, biochemically, the acidic to basic substitution should enhance cleavage by mammalian furin, which is also suggested by an increase in predicted PiTou scores (FIG. 3, panel A). DV1-WT and DV1-prM-D89K displayed no significant difference in virus growth kinetics in Vero (mammalian) and C6/36 (insect) cells (FIG. 3, panel B). In both cell types, DV1-prM-D89K was more mature than DV1-WT, phenocopying the Vero-furin grown DV1-WT (FIG. 3, panel C). In DENV2, while growth kinetic were similar in insect cells, the DV2-prM-E89K variant displayed slightly reduced growth in mammalian cells compared to DV2-WT. Again, DV2-prM-E89K is more mature than DV2-WT in both mammalian and insect cells.

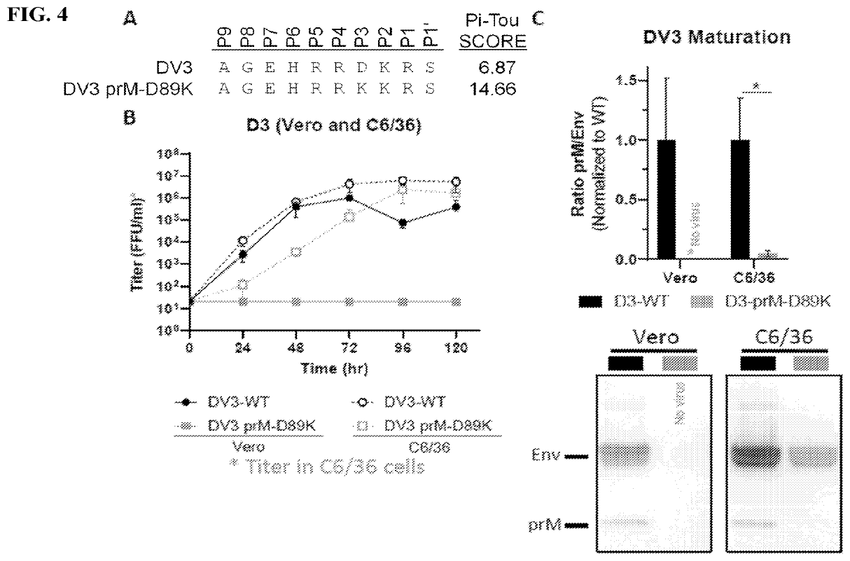

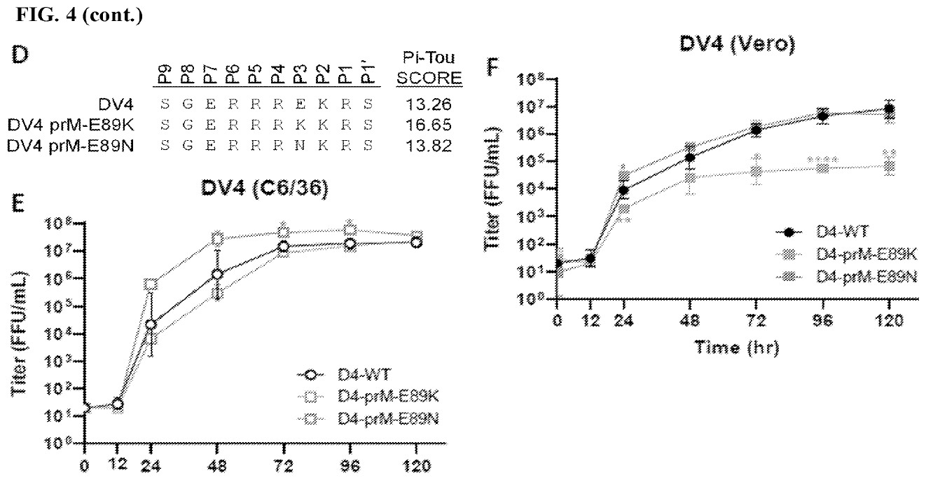

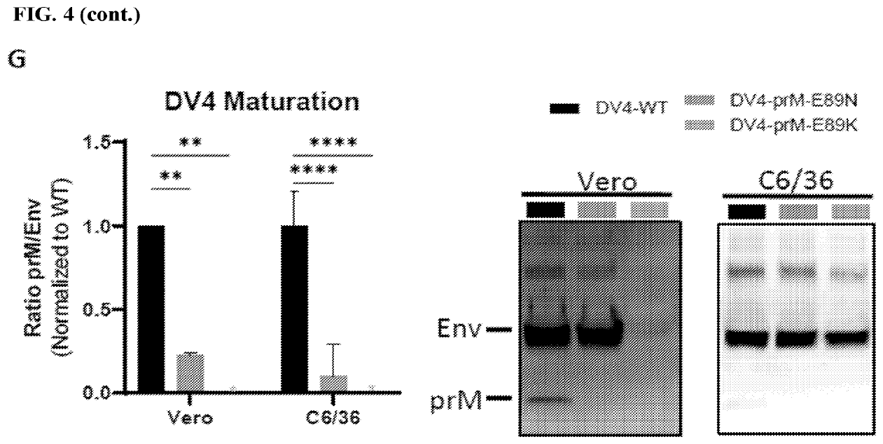

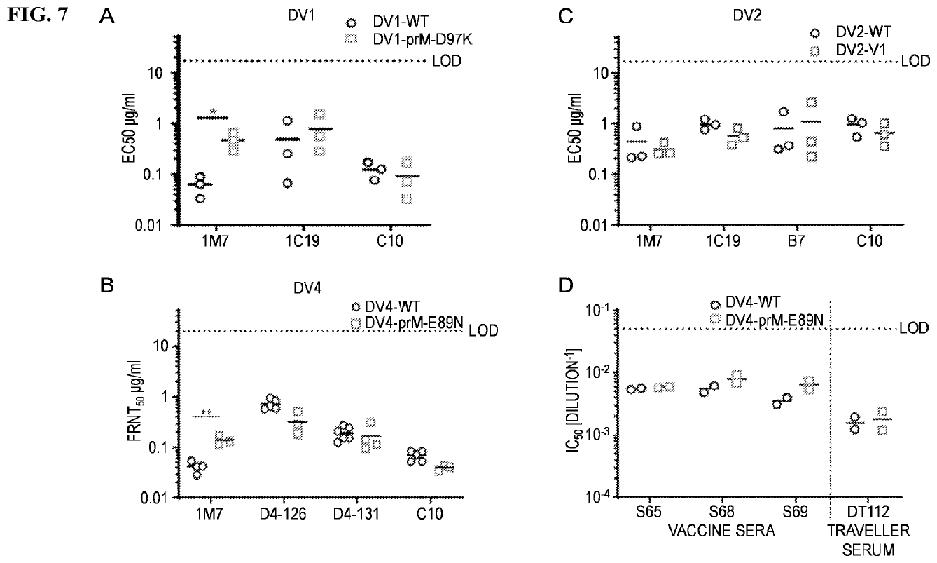

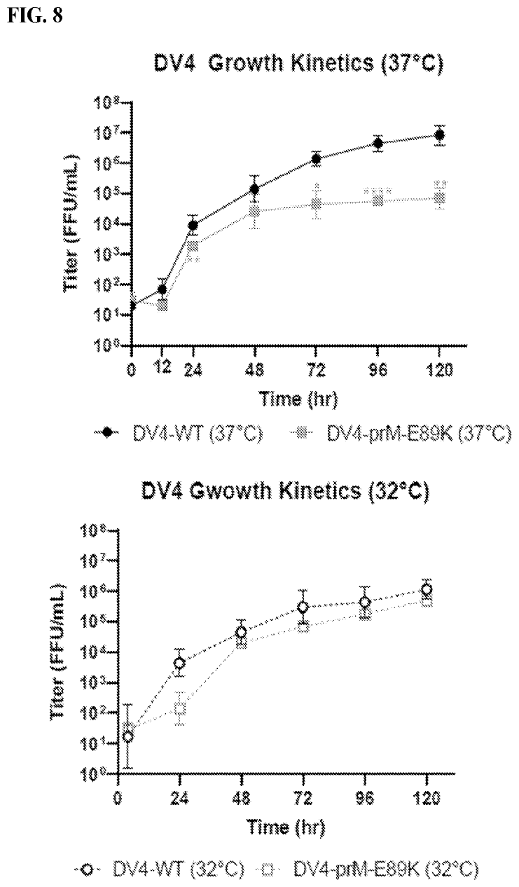

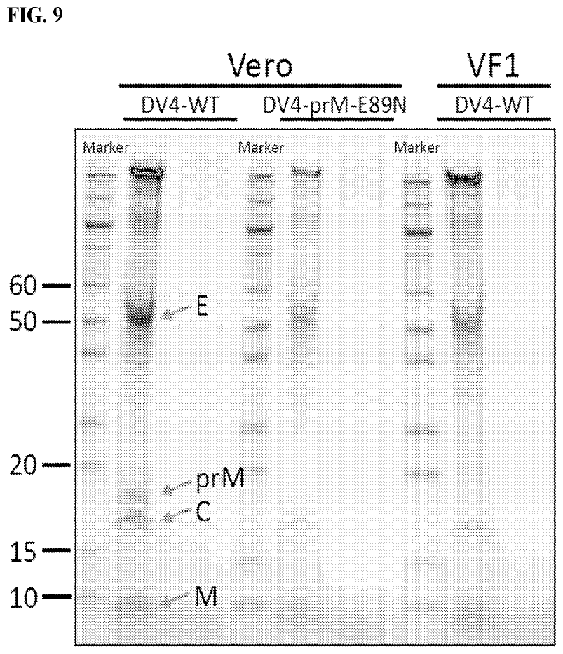

Generation of Mature DENV3 and DENV4 Requires Iterative Optimization of the prM Cleavage Sites. We introduced a similar acidic to basic mutation on the DV3-WT backbone, generating the isogenic strain DV3-prM-D89K (FIG. 4, panel A). While the growth kinetics between DV3-WT and DV3-prM-D89K were similar in insect cells (FIG. 4, panel B), the DV3-prM-D89K variant was unable to grow on mammalian cells (FIG. 4, panel B), and the viruses were compared using C6/36 titers. We could not detect any uncleaved prM protein by Western blot, indicating that DV3-prM-D89K is fully mature (FIG. 4, panel C). Lastly, we introduced the same acidic to basic mutation on the DV4-WT backbone, generating the isogenic strain DV4-prM-E89K (FIG. 4, panel D). Similar to DV3 mutants, the growth kinetics between DV4-WT and DV4-prM-E89K were similar in insect cells (FIG. 4, panel E); however, the DV4-prM-E89K variant displayed a 2-log growth defect compared to DV4-WT on mammalian cells (FIG. 4, panel F). Interestingly, this defect was alleviated when virus was grown at 32° C., suggesting a stability issue of the variant (FIG. 8). A spontaneous mutation, K89N, rapidly emerged in the mutant virus by passage 2. By the 5^{th} passage, the DV4-prM-E89N variant represented 100% of the viral population, supporting the notion that viruses encoding the E89K mutation were out-competed by the E89N mutation in mammalian cells. We re-established DV4-prM-E89N via reverse genetics and, as expected, DV4-prM-E89N had similar growth kinetics to DV4-WT in both Vero and C6/36 cells. The maturations of both variants was tested in comparison to DV4-WT. No prM can be detected in DV4-prM-E89K; due to the low virus yield in mammalian cells, the data suggest that either DV4-prM-E89K is fully mature or the protein input is below detection limit. DV4-prM-E89N is also more mature than DV4-WT in both insect and mammalian cells (FIG. 4, panel G). To look at maturation status more directly, we purified DV4-prM-E89N, DV4-WT, and VF1-grown DV4-WT by sucrose gradient and separated the virions by SDS-Page. Confirming the Western blot results, we detected uncleaved prM in DV4-WT grown in Vero cells, indicating partial immature virions. In contrast, no prM band is present in DV4-prM-E89N or in VF1-grown DV4-WT (FIG. 9).

Based on the biochemical understanding of mammalian furin, the order of substrate favorability is basic (K or R)>neutral (e.g. N)>acidic (D or E) residues. In DENV3 and DENV4, our result indicates that a highly favorable furin cleavage sites (composed of many basic residues) may negatively impact DENV growth in mammalian cells but not insect cells. Alternatively, the extra lysine residue in DENV3 and DENV 4 might pose a specific structural incompatibility in mammalian cells. Nevertheless, at least two rounds of iterative optimization were needed to generate genetically mature DENV4.

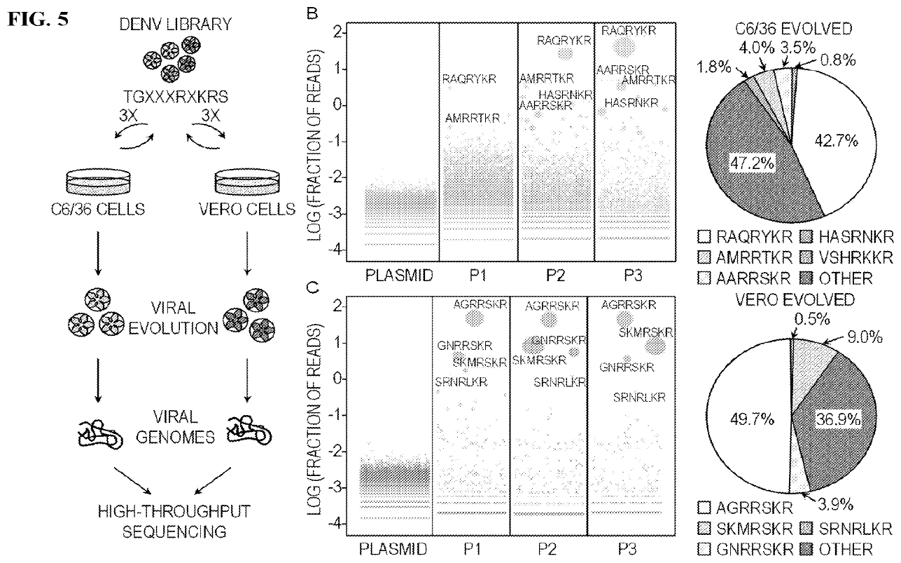

Directed Evolution Reveals High Levels of Plasticity in DENV2 prM Cleavage Site. Our results with DENV3 and DENV4 indicated that the furin cleavage site may play additional roles in viral fitness independent of maturation. Therefore, there may exist optimal sequences of the prM cleavage site for efficient in vitro growth as well as maturation. To test this hypothesis, we selected DV2, the most immature DENV in our panel, which also had reduced viral fitness upon genetic alteration. We performed saturation mutagenesis and directed-evolution to simultaneously screen thousands of DENV2 prM cleavage site variants for efficient growth in mammalian and insect cultures. To ensure the viability of the library, the mutation sites were designed to preserve the minimal core of furin cleavage site, R-X-K-R (FIG. 5, panel A). We selected the P3, P5, P6, and P7 positions of the prM cleavage site for saturation mutagenesis. The viral library was generated and passaged three times in either insect or mammalian cells, and each passage of the virus were deep sequenced along with the plasmid library (FIG. 5, panel A). The theoretical amino acid diversity of the library approached 160,000 variants (excluding stop codons), which was fully represented in the plasmid library (Table 2). As expected, viral diversity rapidly dropped after one passage, to 0.7% (1148 unique variants) and 16.2% (25942 unique variants) of the theoretical maximum in mammalian and insect cells respectively, and further diminished in subsequent passages (Table 2). The relatively large number of viable DENV2 variants in both cells indicated a high degree of plasticity within the prM cleavage site of DENV (Table 2). Interestingly, insect cells were more tolerant to prM cleavage site variations than mammalian cells with 10 times more unique viable variants at the end of passage 3 (Table 2), suggesting a greater range of plasticity in the prM cleavage site.

After three rounds of selection, two different dominant variants, TGRAQRYKR|S (DV2-C1, from insect cells; SEQ ID NO:103) and TGAGRRSKR|S (DV2-V1, from mammalian cells; SEQ ID NO:101), emerged, each representing almost 50% of their respective viral populations (FIG. 5, panel B). Neither variant contained an acidic residue in the proximity (P1-P7) of the furin cleavage site, as opposed to two acidic residues in the WT sequence (FIG. 5, panels B and C). We analyzed the top 50 selected variants from both insect and mammalian cells, and out of 100 variants only four contained an acidic residue (Table 3), showing a general preference to exclude acidic residues at the furin cleavage site in both cell types. Interestingly, there is only one sequence shared between the top 50 variants evolved from insect and mammalian cells after three passages, suggesting host specificity. Although founder effects cannot be excluded in directed-evolution experiments, the clear de-selection of acidic residues and the distinct populations found in insect and mammalian cells despite the shared starting library highlight both the shared and differential selective pressures exerted by the two hosts.

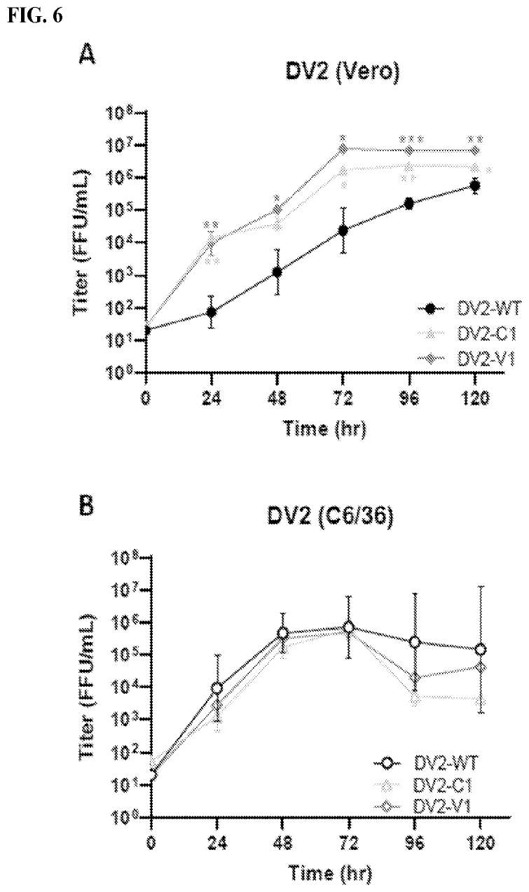

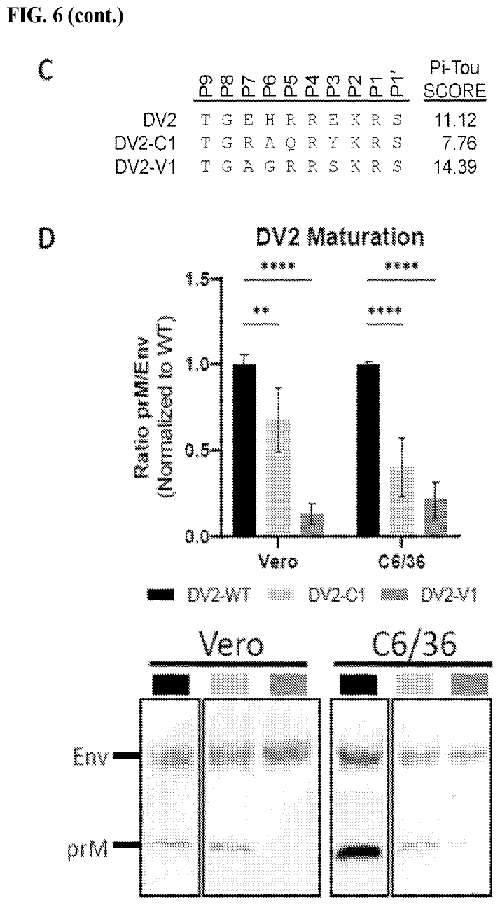

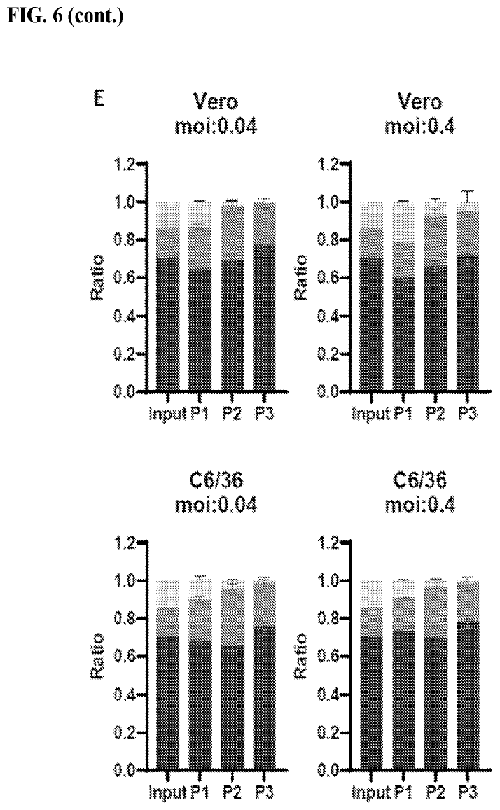

Direct-evolved DENV2 variants are Highly Mature and Produce Higher Yield. The top evolved variants, DV2-V1 and DV2-C1, were re-derived via reverse genetics for further characterization. Both DV2-V1 and DV2-C1 grow to higher titer than DV2-WT in Vero cells, although we observed a slight non-significant drop in titer in C6/36 cells at 96 to 120 hpi (FIG. 6, panels A and B). When we tested maturation status, we found that DV2-V1 is almost fully mature while DV2-C1 is only 30% more mature than DV2-WT when grown in Vero cells (FIG. 6, panel D). When the viruses are grown in C6/36, both variants are 60-70% more mature than DV2-WT (FIG. 6, panel D). To determine the relative fitness of these variants to DV2-WT, we performed competition assays of all three viruses on both Vero and C6/36 cells at low (0.04) and high (0.4) multiplicity of infection (m.o.i.). Although we mixed equal foci forming unit (FFU) of each virus as input, the RNA genome ratio of WT to V1 to C1 is 4.6:1:1, suggesting a different particle to PFU ratio in DV2-WT (FIG. 6, panel E). After three consecutive passages, DV2-C1 was out competed by DV2-WT and DV2-V1 in all conditions tested. In contrast, DV2-V1 was able to maintain its population after 3 passages, displaying similar replicative fitness to DV2-WT.

Impact of Maturation Status on DENV Epitope Presentation and Antigenicity. Once a set of genetically mature DENV viruses had been created, we next evaluated the impact of maturation status on antigenicity. We selected several monoclonal antibodies targeting different regions of the DENV E glycoprotein, including C10 (Envelope-Dimer-Epitope 1), B7 (Envelope-Dimer-Epitope 2), 1C19 (BC loop) and 1M7 (fusion loop). Ab epitopes that are not maturation dependent were preserved, as evidenced by antibodies such as C10, B7, and 1C19 which showed no difference in Foci Reduction Neutralization Titer 50 values (FRNT50) (FIG. 7, panels A to C). However, the fusion loop targeting antibody 1M7 showed significantly different FRNT50 values between fully mature and less mature DENVs in DENV1 and 4, but not in DENV2 (FIG. 7, panels A to C). For DENV4, we also tested polyclonal sera from patients 180 days post DENV4 vaccination or naturally infected patients from a traveler cohort. Polyclonal serum contains a mixture of antibodies which may or may not be affected by virion maturation status. FRNT₅₀ of polyclonal serum was equivalent for fully mature and partially mature DENV4 (FIG. 7, panel D).

In this report, both cell-based and a genetic-based methods were provided to produce fully mature DENVs, and demonstrated the ability to evolve highly fit and mature DENV variants via a single round saturation mutagenesis of the prM furin cleavage site. Although biochemical understanding of mammalian furin and algorithms such as Pi-Tou and ProP could be used as a reference to rationally design optimal furin cleavage site, all predictions are based on mammalian furin rather than invertebrate furin proteases. Human furin and Aedes aegypti furin-like-proteases only share 39% sequence identity and drosophila furin has been shown to have different substrate preferences (Table 5). This may partially explain why Pi-Tou predictions do not correlate as well with the more complex DENV maturation or other viruses that cycle between vertebrate and in vertebrate hosts. Other reasons include cleavage site accessibility, protein structure, stability, and the stem region of prM, which can affect maturation in DENV and other viruses. The degree of maturation can differ among DENV genotypes with identical prM proteins, indicating contributions by an envelope-dependent maturation determinant as well (Gallichotte et al. 2018 Cell Rep. 25:1214-1224). Due to the complicated nature of furin cleavage and maturation in DENV, rational design of furin cleavage site is a process of trial and error. Previous studies mutated the furin cleavage site to generate mature DENV2 as well as dengue virus-like-particles (VLPs) (Shen et al. 2018 Elife 7:1-24; Keelapang et al. 2004 J. Virol. 78:2367-2381). However, replication fitness of the resulting mutants were compromised (Junjhon et al. 2008 J. Virol. 82:107776-10791; Junjhon et al. 2010 J. Virol. 84:8353-8358). Here, we engineered compatible furin cleavage sequences for all four DENV serotypes which generate mature DENV variants with near identical replication fitness.

Substitution of the acidic (D or E) P3 residues with basic (K) residues increased furin cleavage efficiency and resulted in fully mature and replication competent DENV1 and 2. However, the same acidic to basic substitution is not as compatible in DENV 3 and 4, resulting in viruses with growth defects on mammalian cells, which were alleviated when grown at 32° C. In contrast, this growth defect was not observed in DENV grown in VF1 cells, excluding the possibility that fully mature DENV3 and 4 are less fit at 37° C. Possible explanations include impacts on RNA or protein stability/folding, which could be alleviated at a lower temperature. These results hint that the unfavorable acidic residues at P3 may play a structural or regulatory role in DENV fitness. A spontaneous mutation (K89N) in DENV4 restored viral fitness, generating a mature and replication competent DENV4. However, the complete abrogation of viral growth seen in DV3-prM-D98K did not allow for spontaneous mutation. Overall, such stepwise optimization of the furin cleavage site is time consuming, labor intensive and is not guaranteed to be successful.

Using directed-evolution, we tested the fitness of thousands of DENV2 prM cleavage site variants at once and selected for compatible sequences that do not compromise replication fitness. Our data revealed 1) high sequence plasticity of the furin cleavage site, especially in insect cells, 2) more mature DENV is favorable in both cell types, and 3) insect and mammalian cells exert specific selective pressure. We observed ten times more viable variants in insect cells compared to mammalian cells, indicating insects have higher tolerance on mutation in furin sites. This could be intrinsic to the innate immunity and substrate preference of insect vitellogenin/furin, or due to the reduced growth temperature of insect cells at 32° C. Such a high tolerance of mutation in insect cells could drive viral diversity and emergence in nature.

We observed that top Vero- and C6/36-selected variants excluded acidic resides in the cleavage site and are more mature. The selected variants also displayed enhanced growth kinetics as well as a slight increase in peak titer in Vero cells. Our result suggest that experimental evolution specifically selects for more mature DENVs, indicating that a similar strategy can be apply to other serotypes to generate genetically determined mature DENVs.

Cells, plasmids and viruses. Mosquito (Aedes albopictus) C6/36 cells (ATCC #CRL-1660) were maintained in minimum essential medium (MEM) (Gibco) media supplemented with 5% fetal bovine serum (FBS) (HyClone), 100 U/mL penicillin and 100 mg/mL penicillin/streptomycin (P/S) (Gibco), 0.1 mM nonessential amino acids (NEAA) (Gibco), HEPES (Gibco) and 2 mM glutaMAX (Gibco) and incubated in the presence of 5% CO₂ at 32° C. Vero (ATCC #CCL-81), VF-Hi and VF-Lo (generated from this study) were maintained in Dulbecco's Modified Eagle's Medium (DMEM) (Gibco) supplemented with 10% FBS, P/S, NEAA and HEPES and incubate in 5% C02 at 37° C. DENV variants were generated by site-directed mutagenesis using Q5 High-fidelity DNA polymerase (NEB) followed by DENV reverse genetics (see below). The Env and prM of all DENV variants were sequence confirmed. DV1, 2, 3 and 4-WT viruses are grow in C6/36 or Vero cells maintained in infection media. C6/36 infection media contains Opti-MEM (Gibco) supplemented with 2% FBS, 1% P/S, 0.1 mM NEAA, 1% HEPES and 2 mM glutaMAX. Vero infection media is the same as the growth media except with 2% FBS supplement.

DENV reverse genetics. Recombinant viruses were constructed using a four-plasmid cloning strategy. The DENV genome was divided into four fragments (A-D fragment) and subcloned into four separate plasmids. A T7 promoter was introduced into the 5′ end of the A fragment, and unique type IIS restriction endonuclease cleavage sites are introduced into the 5′ and 3′ ends of each fragment to allow for systematic assembly into a full-length cDNA from which the full-length RNA transcripts can be derived. Plasmid DNA was grown in Top10 chemical component cells (ThermoFisher), digested with the corresponding enzymes, gel purified, and ligated together with T4 DNA ligase (NEB). Ligation products were purified by chloroform extraction. The purified ligation product was used as a template for in-vitro transcription to generate infectious genome-length capped viral RNA transcripts using T7 RNA polymerase (ThermoFisher). RNA was electroporated into either C6/36 or Vero cells. Cell culture supernatant containing virus was harvested 4-5 days post-electroporation as passage zero. During the subsequent passages following infection, the cells were grown in infection media.

Stable cell line generation, VeroFurin-Clone-1 (VF1). Human furin was cloned in the sleeping beauty transposon plasmid pSB-bi-RP (Addgene #60513), transfected along with transposase, pCMV(CAT)T7-SB100 (Addgene #34879) into Vero cell using PEI Max (MW 40,000) (Polysciences) and selected with 2.5 μg/ml Puromycin (Gibco). Clonal cell lines were generated through limited dilution of the polyclonal cell line on a 96-well plate at the concentration of 0.3 cell/well.

DENVgrowth kinetic and quantification. 500,000 Vero or C6/36 cells were seeded in each well of a 6-well plate 1 day prior infection. Cells were infected with DENV at 0.05 to 0.1 M.O.I. assuming 1×10⁶ cells on the day of infection. Cells were washed 3 times with PBS and replenished with 3 mL of infection media after 1 hour of inoculation at 37° C. in 5% C02 incubator. 300 μl of viral supernatant was collected and fresh media was replenished at 0, 24, 48, 72, 96 and 120 hpi and stored at −80° C. Titer of the viral supernatant was determined using a standard DENV foci forming assay. In brief, Vero cells were seeded at 2×10⁴ cells/well in a 96-well plate. 50 μl of serial diluted viral supernatant were added to each well and incubated for 1 h at 37° C. in 5% C02 incubator. 125 μl of overlay (Opti-MEM+5% methyl cellulose+NEAA+P/S) was added to each well and incubated for 48 h at 37° C.+5% CO₂. Each well was rinsed 3 times with PBS and fixed with 10% formalin in PBS for staining. Vero cells were blocked in permeabilization buffer (eBioscience) with 5% non-fat dried milk. Two primary antibodies, anti-prM mAb 2H2 and anti-Env mAb 4G2, from non-purified hybridoma supernatant were used at 1:500 dilution in blocking buffer. Goat anti-mouse secondary conjugated with horseradish peroxidase (HRP) (SeraCare's KPL) were diluted at 1:1000 in blocking buffer. Foci were developed using TrueBlue HRP substrate (SeraCare's KPL) and counted using an automated Immunospot Analyzer instrument (Cellular Technology Limited). All experiments were performed independently a minimum of 3 times.

Immunostaining and western blotting for human furin. Cells were fixed in 10% formalin in PBS and permeabilized with permeabilization buffer (eBioscience). Rabbit anti-furin (Thermo, PA1-062, 1:1000) was used as primary antibody. Goat anti-rabbit Alexa488 (Invitrogen, 1:2000) as secondary antibody. For western blotting, cell were lysed in 1% TritonX100, 100 mM Tris, 2M NaCl and 100 mM EDTA. Cell lysates were run in SDS-PAGE and blotted onto PVDF membrane. Furin bands were detected using rabbit anti-furin polyclonal at 1:1000 and Goat anti-rabbit HRP (Invitrogen, 1:5000) was used as secondary antibody.

Western Blotting for DENV maturation. Viral stocks or supernatant from DENV growth curves at 120 hpi were diluted with 4× Laemmli Sample Buffer (Bio-Rad) and boiled at 95° C. for 5 minutes. Following SDS-PAGE electrophoresis, proteins were transferred to PVDF membrane and blocked in blocking buffer consist of 3% non-fat milk in PBS+0.05% Tween-20 (PBS-T). The membrane was incubated with polyclonal rabbit anti-prM (1:1000, Invitrogen, Cat. #PA5-34966) and purified human anti-Env (fusion loop) 1M7 (2 μg/ml) in 2% BSA+PBS-T solution for 1 h at 37° C. The primary antigen-antibody complex was detected by incubating the blot with goat anti-rabbit IgG HRP (1:10000, Jackson-ImmunoLab) and sheep anti-human IgG HRP (1:5000, GE Healthcare) in 3% milk in PBS-T, for 1 h at room temperature. Membranes were developed by Supersignal West Pico PLUS Chemiluminescent Substrate (ThermoFisher). Western blot images were captured with iBright FL1500 imaging system (Invitrogen). The pixel intensity of individual bands was measured using ImageJ, and relative maturation was calculated by using the following equation: (prM_{Exp}/Env_{Exp})/(prM_{WT}/Env_{WT}). All experiments were performed independently a minimum of 3 times.

Foci reduction neutralization titer assay (FRNT Assay). FRNT assays were performed on Vero cells. Briefly, 2×10⁴ Vero cells were seeded in a 96-well plate. Antiserum or mAbs were serially diluted and mixed with DENV viruses (80-100 FFU/well) at a 1:1 volume ratio and incubated at 37° C. for 1 h without the cells. The mixture was transferred to the 96-well plate with Vero cells and incubated at 37° C. for 1 h. The plate is subsequently overlaid with overlay medium (see above). Viral foci were stained and counted as described above. Data were fitted with variable slope sigmoidal dose-response curves and FRNT₅₀ were calculated with top or bottom restraints of 100 and 0, respectively. All experiments were performed independently at least 2 times, due to limited amounts of human serum.

DENV2 library generation and directed-evolution. DENV prM libraries were engineered through saturation mutagenesis on amino acid residues P3, 5, 6 and 7 of the DENV furin cleavage site. In brief, degenerate NNK oligos (Integrated DNA Technologies) were used to amplify the prM region to generate a library with mutated prM DNA fragments. To limit bias and ensure accuracy, Q5 high fidelity polymerase (NEB) was used and limited to <18 cycles of amplification. The DNA library was cloned into the DENV reverse genetics system plasmid A to create a plasmid library by standard restriction digestion. Ligation reactions were then concentrated and purified by ethanol precipitation. Purified ligation products were electroporated into DH10B ElectroMax cells (Invitrogen) and directly plated on multiple 5,245-mm² bioassay dishes (Corning) to avoid bias from bacterial suspension cultures. Colonies were pooled and purified using a Maxiprep Kit (Qiagen). The plasmid library was used for DENV reverse genetics as described above. The in vitro transcribed DENV RNA library was electroporated in either Vero or C6/36 cells, the viral supernatants were passaged 3 times every 4 to 5 days in the corresponding cells for enrichment.

High-throughput sequencing and analysis. Viral RNA was isolated using a QIAamp Viral RNA Mini Kit (Qiagen). Amplicons containing the library regions were prepared for sequencing through two rounds of PCR, using the Illumina TruSeq system and Q5 Hot Start DNA polymerase (NEB). Primers for the first round of PCR were specific to the DENV2 prM sequence with overhangs for Illumina adapters. This PCR product was purified and used as a template for a second round of PCR using the standard Illumina P5 and P7 primers with barcodes and sequencing adaptors. PCR products were purified and analyzed on a Qubit 4 fluorometer (Invitrogen) and Bioanalyzer (Agilent Technologies) for quality control. Amplicon libraries were diluted to 4 nM and pooled for sequencing, which was carried out on a MiSeq system with 300 bp paired-end reads. Plasmid and P0 libraries were sequenced at a depth of ˜1 million reads per sample; further passages were sequenced with depth between 300,000-1 million reads to sample. A custom perl script (Tse et al. 2017 PNAS 114:E4812-E4821) was used to analyze the sequences, and custom R scripts were used to plot the data.

DENV competition assay. DV2-WT, DV2-V1 and DV2-C1 were mixed at 1:1:1 FFU ratio and used to infect Vero or C6/36 cells at two different M.O.I. (0.4 or 0.04) in a 6-well plate. At 4 dpi, supernatants were collected and titered before the next round of infection for a total of 3 passages. Viral RNA was isolated from each passage using the Maxwell RSC48 (Promega) automated RNA extraction machine. Viral cDNA were generated using SuperScript IV reverse transcriptase (Invitrogen) using universal reverse primer 5′-CTRATYTCCATSCCRTACCAGC-3′ (SEQ ID NO:129). To quantify the relative amount of each variant in the population, we developed a multiplex assay using digital droplet PCR (ddPCR) using general DENV2 probe (HEX) and specific cleavage site probes (FAM) (Table 4).

Purification of DENV by sucrose gradient ultracentrifugation. Five ridged bottles of Vero or VF-1 (70-80% confluent) were infected with DV4-WT or DV4-prM-E89N at 0.01 M.O.I. in DMEM/F12 media supplemented with 2% FBS for 3 days. Infection media was replaced with DMEM/F12 serum free media and virus supernatant was harvested 2 days and 4 days after the media change. Supernatants were clarified by filtration via 0.45 μm filter. Filtrates were pooled together and concentrated via tangential flow filtration using Pellicon2 Mini Cassette with Biomax 100 kDa membrane (Millipore) from 1 Liter to 45 ml. Concentrated supernatant were purified via 8-step (60, 55, 50, 45, 40, 35, 30 and 15%) sucrose gradient ultracentrifugation with 22 ml of supernatant in each tube spinning at 17,000 rpm for 18 h at 4° C. Fractions (1 ml) were collected and run on a TGX stain-free gel (Bio-rad).

Furin cleavage prediction. Furin cleavage site efficiency was predicted using the Pi-Tou software (Tian et al. 2012 Sci Rep. 2), providing amino acids from position P14-P6′ of the DENV furin cleavage sites.

Statistical analysis. Statistical analysis was carried out using Graphpad Prism version 9.0. Growth kinetics and maturation of DENV variants were compared to their corresponding wildtype using 2-way ANOVA multiple comparisons. Neutralization titers of DENV variants were compared to their corresponding wildtype using Student's t-test. Significance symbols are defined as follow: * p<0.05, ** p<0.01, *** p<0.001, **** p<0.0001. Data are graphed as mean +/−standard deviation.

TABLE 1
DENV prototypes.

| Serotype | Strain | Genotype |
|---|---|---|
| DV1 | WestPac 74 (WHO) | IV |
| DV2 | S16803 (WHO) | Asian I |
| DV3 | 3001 | III |
| DV4 | Sri Lanka 92 | IIb |

TABLE 2
Summary of plasmids and passages diversities
of DV2 directed-evolution.

| | |
|---|---|
| C6/36 Evolved | Vero Evolved |

| | Unique | % | Unique | % |
|---|---|---|---|---|
| | Sequences | Maximum | Sequences | Maximum |
| Plasmid | 164569 | 102.86* | 164569 | 102.86* |
| P1 | 25942 | 16.21 | 1148 | 0.72 |
| P2 | 14119 | 8.82 | 719 | 0.45 |
| P3 | 6026 | 3.77 | 683 | 0.43 |

TABLE 3
Top 50 enriched sequences and PiTou scores
of DV2 directed-evolution.

| | | | SEQ | | | | SEQ |
|---|---|---|---|---|---|---|---|
| | | Pi-Tou | ID | | | Pi-Tou | ID |
| Name | Sequence | Score | NO | Name | Sequence | Score | NO |
| DENV- | TGQNSRLK | 11.2574 | 1 | DENV- | TGMAKRSK | 13.7489 | 51 |
| DC1 | RS | | | DV1 | RS | | |
| DENV- | TGQMSRN | 8.01577 | 2 | DENV- | TGTAKRSK | 13.7489 | 52 |
| DC2 | KRS | | | DV2 | RS | | |
| DENV- | TGSNYRSK | 4.65351 | 3 | DENV- | TGLSRRSK | 15.3795 | 53 |
| DC3 | RS | | | DV3 | RS | | |
| DENV- | TGLFTRNK | 6.15277 | 4 | DENV- | TGRQARSK | 11.0661 | 54 |
| DC4 | RS | | | DV4 | RS | | |
| DENV- | TGRLRRAK | 12.1042 | 5 | DENV- | TGKMRREK | 8.66191 | 55 |
| DC5 | RS | | | DV5 | RS | | |
| DENV- | TGTPKRLK | 13.0894 | 6 | DENV- | TGSNKRHK | 10.7461 | 56 |
| DC6 | RS | | | DV6 | RS | | |
| DENV- | TGKINRAK | 2.98847 | 7 | DENV- | TGERARVK | 12.8337 | 57 |
| DC7 | RS | | | DV7 | RS | | |
| DENV- | TGSFTRSK | 8.43697 | 8 | DENV- | TGRYKRDK | 4.10841 | 58 |
| DC8 | RS | | | DV8 | RS | | |
| DENV- | TGGSPRAK | 10.126 | 9 | DENV- | TGGKSRVK | 13.5558 | 59 |
| DC9 | RS | | | DV9 | RS | | |
| DENV- | TGSKLRIK | 11.3193 | 10 | DENV- | TGRPVRSK | 12.6207 | 60 |
| DC10 | RS | | | DV10 | RS | | |
| DENV- | TGTGTRLK | 10.9346 | 11 | DENV- | TGHSRREK | 12.5334 | 61 |
| DC11 | RS | | | DV11 | RS | | |
| DENV- | TGHMNRLK | 7.79803 | 12 | DENV- | TGWGKRSK | 13.7489 | 62 |
| DC12 | RS | | | DV12 | RS | | |
| DENV- | TGFSTRQK | 11.8901 | 13 | DENV- | TGTGRRMK | 12.5588 | 63 |
| DC13 | RS | | | DV13 | RS | | |
| DENV- | TGGTTRAK | 9.63526 | 14 | DENV- | TGRSKRSK | 14.2854 | 64 |
| DC14 | RS | | | DV14 | RS | | |
| DENV- | TGESMRSK | 11.0639 | 15 | DENV- | TGSVRRVK | 12.5076 | 65 |
| DC15 | RS | | | DV15 | RS | | |
| DENV- | TGYRSRPK | 13.0114 | 16 | DENV- | TGASHRSK | 13.016 | 66 |
| DC16 | RS | | | DV16 | RS | | |
| DENV- | TGSNSRAK | 9.87178 | 17 | DENV- | TGMSKRTK | 14.4649 | 67 |
| DC17 | RS | | | DV17 | RS | | |
| DENV- | TGRSIRSK | 12.8553 | 18 | DENV- | TGFKHRVK | 12.2057 | 68 |
| DC18 | RS | | | DV18 | RS | | |
| DENV- | TGHDSRHK | 9.20017 | 19 | DENV- | TGGRHRNK | 11.2579 | 69 |
| DC19 | RS | | | DV19 | RS | | |
| DENV- | TGFVGRHK | 9.53267 | 20 | DENV- | TGATKRSK | 13.8734 | 70 |
| DC20 | RS | | | DV20 | RS | | |
| DENV- | TGGAHRLK | 11.6078 | 21 | DENV- | TGISKRGK | 11.1775 | 71 |
| DC21 | RS | | | DV21 | RS | | |
| DENV- | TGSNPRMK | 8.12156 | 22 | DENV- | TGNHRRNK | 12.1042 | 72 |
| DC22 | RS | | | DV22 | RS | | |
| DENV- | TGSNTRIK | 9.01827 | 23 | DENV- | TGSLRRIK | 13.0807 | 73 |
| DC23 | RS | | | DV23 | RS | | |
| DENV- | TGVTARTK | 12.808 | 24 | DENV- | TGQYKRSK | 11.3468 | 74 |
| DC24 | RS | | | DV24 | RS | | |
| DENV- | TGTRVRSK | 13.5421 | 25 | DENV- | TGRPRRDK | 7.84358 | 75 |
| DC25 | RS | | | DV25 | RS | | |
| DENV- | TGHVGRDK | 3.86024 | 26 | DENV- | TGYSKRPK | 12.4046 | 76 |
| DC26 | RS | | | DV26 | RS | | |
| DENV- | TGSIMRHK | 2.208 | 27 | DENV- | TGMAQRS | 11.1173 | 77 |
| DC27 | RS | | | DV27 | KRS | | |
| DENV- | TGPKSRLK | 13.5558 | 28 | DENV- | TGTSRRNK | 13.0953 | 78 |
| DC28 | RS | | | DV28 | RS | | |
| DENV- | TGNVRRYK | 9.62714 | 29 | DENV- | TGQKARSK | 13.2943 | 79 |
| DC29 | RS | | | DV29 | RS | | |
| DENV- | TGYSRRDK | 8.14108 | 30 | DENV- | TGIAKRSK | 13.4871 | 80 |
| DC30 | RS | | | DV30 | RS | | |
| DENV- | TGYGHRYK | 8.72497 | 31 | DENV- | TGGRTROK | 12.6208 | 81 |
| DC31 | RS | | | DV31 | RS | | |
| DENV- | TGAANRLK | 11.1661 | 32 | DENV- | TGRKVRSK | 13.1042 | 82 |
| DC32 | RS | | | DV32 | RS | | |
| DENV- | TGVHNRNK | 9.49747 | 33 | DENV- | TGSMKRSK | 10.4139 | 83 |
| DC33 | RS | | | DV33 | RS | | |
| DENV- | TGVTARLK | 11.73 | 34 | DENV- | TGTAQRSK | 11.1173 | 84 |
| DC34 | RS | | | DV34 | RS | | |
| DENV- | TGHNTRDK | 3.08753 | 35 | DENV- | TGNTHRTK | 12.7816 | 85 |
| DC35 | RS | | | DV35 | RS | | |
| DENV- | TGASHRPK | 11.1351 | 36 | DENV- | TGGFRRYK | 7.72902 | 86 |
| DC36 | RS | | | DV36 | RS | | |
| DENV- | TGGTRRVK | 14.0689 | 37 | DENV- | TGNKSRNK | 12.1701 | 87 |
| DC37 | RS | | | DV37 | RS | | |
| DENV- | TGVPMRQ | 10.3953 | 38 | DENV- | TGKTRRDK | 7.72902 | 88 |
| DC38 | KRS | | | DV38 | RS | | |
| DENV- | TGSGNRAK | 9.77666 | 39 | DENV- | TGMTRRGK | 12.1213 | 89 |
| DC39 | RS | | | DV39 | RS | | |
| DENV- | TGKIGREK | 2.90178 | 40 | DENV- | TGRHRRDK | 7.15001 | 90 |
| DC40 | RS | | | DV40 | RS | | |
| DENV- | TGHHNRTK | 11.9611 | 41 | DENV- | TGTSRRHK | 13.8135 | 91 |
| DC41 | RS | | | DV41 | RS | | |
| DENV- | TGWTARSK | 12.6286 | 42 | DENV- | TGQNRRDK | 6.16771 | 92 |
| DC42 | RS | | | DV42 | RS | | |
| DENV- | TGSKIRDK | 5.86584 | 43 | DENV- | TGMAKRTK | 13.9284 | 93 |
| DC43 | RS | | | DV43 | RS | | |
| DENV- | TGLHGRPK | 10.3586 | 44 | DENV- | TGTAKRLK | 12.8504 | 94 |
| DC44 | RS | | | DV44 | RS | | |
| DENV- | TGTAGRNK | 10.2558 | 45 | DENV- | TGSLSRHK | 11.6436 | 95 |
| DC45 | RS | | | DV45 | RS | | |
| DENV- | TGDQRRLK | 12.5076 | 46 | DENV- | TGLSKRSK | 14.2854 | 96 |
| DC46 | RS | | | DV46 | RS | | |
| DENV- | TGRTHRFK | 10.318 | 47 | DENV- | TGASRRDK | 8.14108 | 97 |
| DC47 | RS | | | DV47 | RS | | |
| DENV- | TGGFGRSK | 9.14369 | 48 | DENV- | TGSKNRAK | 10.601 | 98 |
| DC48 | RS | | | DV48 | RS | | |
| DENV- | TGHSYRPK | 4.78077 | 49 | DENV- | TGSRVRDK | 6.30372 | 99 |
| DC49 | RS | | | DV49 | RS | | |
| DENV- | TGERWRHK | −5.40603 | 50 | DENV- | TGDTRRSK | 14.9674 | 100 |
| DC50 | RS | | | DV50 | RS | | |

TABLE 4

| | | SEQ |
|---|---|---|
| | | ID |
| Name | Sequences | NO |
| DV2-C1-F | GTAACTTATGGGACTTGTACTACCAC | 117 |
| DV2-C1-Probe- | AGCGCTTATATCTCTGAGCACGTCC | 118 |
| FAM | | |
| DV2-C1-R | CACATGTGGAACGAGTGC | 119 |
| DV2-V1-F | GTAACTTATGGGACTTGTACTACCAC | 120 |
| DV2-V1-Probe- | CACTGAGCGCTTACTICTACGCCC | 121 |
| FAM | | |
| DV2-V1-R | CACATGTGGAACGAGTGC | 122 |
| DV2-WT-F | TGGGTAACTTATGGGACTTGTA | 123 |
| DV2-WT-Probe- | ACCACGGGAGAACATAGAAGAGAA | 124 |
| FAM | | |
| DV2-WT-R | CACATGTGGAACGAGTGC | 125 |
| DV2-total-F | CACCATAATGGCAGCAATCC | 126 |
| DV2-total- | ACGACACATTTCCAGAGAGCCCTG | 127 |
| Probe-Hex | | |
| DV2-total-R | ACAGCTGTCAGTAAGATGAAA | 128 |

TABLE 5
% Identity (amino acid).

| | | | Aedes- | | |
|---|---|---|---|---|---|
| | | Aedes-furin-like | Vitellogenin | Aedes-Furin-like | Aedes- |
| | Human-Furin | protease 1 | convertase | protease 2 | AAEL010725-PA |
| Human-Furin | | 39% | 39% | 39% | 33% |
| Aedes-furin-like protease 1 | 39% | | 99% | 42% | 34% |
| Aedes-Vitellogenin convertase | 39% | 99% | | 42% | 34% |
| Aedes-Furin-like protease 2 | 39% | 42% | 42% | | 98% |
| Aedes-AAEL010725-PA | 33% | 34% | 34% | 98% | |

TABLE 6

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

TABLE 7

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,2′-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | aIle |
| Methionine sulfoxide | MSO |
| N-Methylglycine, sarcosine | MeGly |
| N-Methylisoleucine | MeIle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-Cl) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

ADDITIONAL SEQUENCES
[DV2-V1]
SEQ ID NO: 101.
TGAGRRSKRS
[DV2-V2]
SEQ ID NO: 102.
TGSKMRSKRS
[DV2-C1]
SEQ ID NO: 103.
TGRAQRYKRS
[DV2-prM] GenBank: GU289914.1
SEQ ID NO: 104.
FHLTTRNGEPHMIVSRQEKGKSLLFKTEDGVNMCTLMAMDLGE
LCEDTITYNCPLLRQNEPEDIDCWCNSTSTWVTYGTCTTTGEH
RREKRSVALVPHVGMGLETRTETWMSSEGAWKHAQRIETWILR
HPGFTIMAAILAYTIGTTHFQRALIFILLTAVAPSMT
[DV2-prM]
SEQ ID NO: 105.
TTCCATCTAACCACACGTAACGGAGAACCACACATGATCGTCA
GTAGACAAGAGAAAGGGAAAAGTCTTCTGTTTAAAACAGAGGA
TGGTGTGAACATGTGCACCCTCATGGCCATGGACCTTGGTGAA
TTGTGTGAAGACACAATCACGTACAACTGTCCCCTTCTCAGGC
AGAATGAGCCAGAAGACATAGACTGTTGGTGCAACTCCACGTC
CACATGGGTAACTTATGGGACTTGTACTACCACGGGAGAACAT
AGAAGAGAAAAAAGATCAGTGGCACTCGTTCCACATGTGGGAA
TGGGACTGGAGACGCGAACTGAAACATGGATGTCATCAGAAGG
GGCTTGGAAACATGCCCAGAGAATTGAAACTTGGATCCTGAGA
CATCCAGGCTTCACCATAATGGCAGCAATCCTGGCATATACCA
TAGGGACGACACATTTCCAGAGAGCCCTGATTTTCATCTTACT
GACAGCTGTCGCTCCTTCAATGACA
[DV1-prM] GenBank: U88535.1
SEQ ID NO: 106.
FHLTTRGGEPHMIVSKQERGKSLLFKTSAGVNMCTLIAMDLGE
LCEDTMTYKCPRITETEPDDVDCWCNATETWVTYGTCSQTGEH
RRDKRSVALAPHVGLGLETRTETWMSSEGAWKQIQKVETWALR
HPGFTVIALFLAHAIGTSITQKGIIFILLMLVTPSMA
[DV3-prM] GenBank: JQ411814
SEQ ID NO: 107.
FHLTSRDGEPRMIVGKNERGKSLLFKTASGINMCTLIAMDLGE
MCDDTVTYKCPHITEVEPEDIDCWCNLTSTWVTYGTCNQAGEH
RRDKRSVALAPHVGMGLDTRTQTWMSAEGAWRQVEKVETWALR
HPGFTILALFLAHYIGTSLTQKVVIFILLMLVTPSMT
[DV4-prM] GenBank: KJ160504.1
SEQ ID NO: 108.
FHLSTRDGEPLMIVAKHERGRPLLFKTTEGINKCTLIAMDLGE
MCEDTVTYKCPLLVNTEPEDIDCWCNLTSTWVMYGTCTQSGER
RREKRSVALTPHSGMGLETRAETWMSSEGAWKHAQRVESWILR
NPGFALLAGFMAYMIGQTGIQRTVFFVLMMLVAPSYG
[DV2-V2 prM]
SEQ ID NO: 109.
FHLTTRNGEPHMIVSRQEKGKSLLFKTEDGVNMCTLMAMDLGE
LCEDTITYNCPLLRQNEPEDIDCWCNSTSTWVTYGTCTTTGSK
MRSKRSVALVPHVGMGLETRTETWMSSEGAWKHAQRIETWILR
HPGFTIMAAILAYTIGTTHFQRALIFILLTAVAPSMT
DV2-C1 prM
SEQ ID NO: 110.
FHLTTRNGEPHMIVSRQEKGKSLLFKTEDGVNMCTLMAMDLGE
LCEDTITYNCPLLRQNEPEDIDCWCNSTSTWVTYGTCTTTGRA
QRYKRSVALVPHVGMGLETRTETWMSSEGAWKHAQRIETWILR
HPGFTIMAAILAYTIGTTHFQRALIFILLTAVAPSMT
DV2-V1 prM
SEQ ID NO: 111.
FHLTTRNGEPHMIVSRQEKGKSLLFKTEDGVNMCTLMAMDLGE
LCEDTITYNCPLLRQNEPEDIDCWCNSTSTWVTYGTCTTTGAG
RRSKRSVALVPHVGMGLETRTETWMSSEGAWKHAQRIETWILR
HPGFTIMAAILAYTIGTTHFQRALIFILLTAVAPSMT
DV1-prM-D89K
SEQ ID NO: 112.
FHLTTRGGEPHMIVSKQERGKSLLFKTSAGVNMCTLIAMDLGE
LCEDTMTYKCPRITETEPDDVDCWCNATETWVTYGTCSQTGEH
RRKKRSVALAPHVGLGLETRTETWMSSEGAWKQIQKVETWALR
HPGFTVI ALFLAHAIGTSITQKGIIFILLMLVTPSMA
DV2-prM-E89K
SEQ ID NO: 113.
FHLTTRNGEPHMIVSRQEKGKSLLFKTEDGVNMCTLMAMDLGE
LCEDTITYNCPLLRQNEPEDIDCWCNSTSTWVTYGTCTTTGEH
RRKKRSVALVPHVGMGLETRTETWMSSEGAWKHAQRIETWILR
HPGFTIMAAILAYTIGTTHFQRALIFILLTAVAPSMT
DV3-prM-E89K
SEQ ID NO: 114.
FHLTSRDGEPRMIVGKNERGKSLLFKTASGINMCTLIAMDLGE
MCDDTVTYKCPHITEVEPEDIDCWCNLTSTWVTYGTCNQAGEH
RRKKRSVALAPHVGMGLDTRTQTWMSAEGAWRQVEKVETWALR
HPGFTILALFLAHYIGTSLTQKVVIFILLMLVTPSMT
DV4-prM-E89K
SEQ ID NO: 115.
FHLSTRDGEPLMIVAKHERGRPLLFKTTEGINKCTLIAMDLGE
MCEDTVTYKCPLLVNTEPEDIDCWCNLTSTWVMYGTCTQSGER
RRKKRSVALTPHSGMGLETRAETWMSSEGAWKHAQRVESWILR
NPGFALLAGFMAYMIGQTGIQRTVFFVLMMLVAPSYG
DV4-prM-E89N
SEQ ID NO: 116.
FHLSTRDGEPLMIVAKHERGRPLLFKTTEGINKCTLIAMDLGE
MCEDTVTYKCPLLVNTEPEDIDCWCNLTSTWVMYGTCTQSGER
RRNKRSVALTPHSGMGLETRAETWMSSEGAWKHAQRVESWILR
NPGFALLAGFMAYMIGQTGIQRTVFFVLMMLVAPSYG

The foregoing is illustrative of the present invention, and is not to be construed as limiting thereof. The invention is defined by the following claims, with equivalents of the claims to be included therein.

1. A recombinant flavivirus prM glycoprotein comprising a prM glycoprotein backbone and at least one amino acid substitution at position 89, 87, 86, and/or 85 in a furin cleavage site, wherein the numbering corresponds to SEQ ID NO:104, and wherein the at least one amino acid substitution introduces an amino acid residue in the furin cleavage site that is more basic than the original amino acid residue, and wherein the furin cleavage site has enhanced cleavability by a furin enzyme.

2. The recombinant flavivirus prM glycoprotein of claim 1, wherein the furin cleavage site comprises an amino acid substitution at position 89.

3. (canceled)

4. The recombinant flavivirus prM glycoprotein of claim 1, wherein the substitution of an amino acid residue that is more basic comprises 89K, 89N, 89Y, or 89S.

5-7. (canceled)

8. The recombinant flavivirus prM glycoprotein of claim 1, comprising one or more amino acid substitutions at positions 87, 86, and/or 85 of the backbone prM glycoprotein, wherein the one or more amino acid substitutions at positions 87, 86, and 85 comprise 85R, 85A, 85S, 86A, 86G, 86K, 87Q, 87R, 87M, or any combination thereof.

9-11. (canceled)

12. The recombinant flavivirus prM glycoprotein of claim 14, wherein the furin cleavage site cleavability is about 1.5-fold enhanced as measured by relative Pi-Tou score of the substituted furin cleavage site as compared to the Pi-Tou score of an unsubstituted furin cleavage site in a corresponding wildtype flavivirus prM glycoprotein, and/or wherein the furin cleavage site has a Pi-Tou score of about 5 or higher.

13. (canceled)

14. The recombinant flavivirus prM glycoprotein of claim 1, comprising the substitution of the amino acid sequence of any one of SEQ ID NO:1-103 in amino acid positions 83-92.

15. The recombinant flavivirus prM glycoprotein of claim 1, wherein the prM glycoprotein backbone comprises a backbone of a dengue virus (DENV, also referred to as DV), zika virus (ZIKV), West Nile virus (WNV), Japanese encephalitis virus (JEV), yellow fever virus (YFV), tick-borne encephalitis virus (TBEV), Kyasanur Forest disease virus (KFDV), Kunjin virus (KUN), Murray Valley encephalitis virus (MVEV), St. Louis encephalitis virus (SLEV), Powassan virus (POW), Usutu virus (USUV), and/or any derivative thereof.

16. (canceled)

17. The recombinant flavivirus prM glycoprotein of claim 1, comprising an amino acid sequence at least 90% identical to any one of the amino acid sequence of SEQ ID NO:109-116.

18. An isolated nucleic acid molecule encoding the flavivirus prM glycoprotein of claim 1.

19. The isolated nucleic acid molecule of claim 18, comprising an mRNA molecule.

20. A flavivirus particle or virus like particle (VLP) comprising the prM glycoprotein of claim 1.

21-23. (canceled)

24. A vector comprising the isolated nucleic acid molecule of claim 18.

25. A population of flavivirus particles comprising the flavivirus particle of claim 20.

26-28. (canceled)

29. A composition comprising the recombinant prM glycoprotein of claim 1, in a pharmaceutically acceptable carrier.

30. A method of producing an immune response to a flavivirus in a subject, comprising administering to the subject an effective amount of the recombinant prM glycoprotein of claim 1.

31. A method of treating a flavivirus infection in a subject, comprising administering to the subject an effective amount of the recombinant prM glycoprotein of claim 1.

32. (canceled)

33. A method of protecting a subject from the effects of a flavivirus infection, comprising administering to the subject an effective amount of the recombinant prM glycoprotein of claim 1.

34-38. (canceled)

39. A method of producing a mature flavivirus particle, comprising:
constructing a flavivirus particle comprising the substituted prM glycoprotein of claim 1, wherein the presence of the substituted prM glycoprotein induces the flavivirus particle to assemble in a mature form;
thereby producing a mature flavivirus particle.

40. (canceled)

41. A method of enhancing antigenicity of a flavivirus particle, comprising:
constructing a flavivirus particle comprising the substituted prM glycoprotein of claim 1, wherein the presence of the substituted prM glycoprotein induces the flavivirus particle to assemble in a mature form;
thereby enhancing the antigenicity of the flavivirus particle.

42. A method of enhancing infectivity of a flavivirus particle, comprising:
constructing a flavivirus particle comprising the substituted prM glycoprotein of claim 1, wherein the presence of the substituted prM glycoprotein induces the flavivirus particle to assemble in a mature form;
thereby enhancing the infectivity of the flavivirus particle.

43-46. (canceled)
